(19) United States
(12) Patent Application Publication
(10) Pub. No.: US 2026/0265783 A1
(43) Pub. Date: Sep. 10, 2026

(54) Strategies For Knock-Ins At AAVS1 Safe Harbor Sites

(71) Applicant: EmendoBio Inc., Wilmington, DE (US)

(72) Inventors: Rafi EMMANUEL, Ramla (IL); Liat ROCKAH, Rishon LeZion (IL)

(73) Assignee: EmendoBio Inc., Wilmington, DE (US)

(21) Appl. No.: 19/167,667

(22) PCT Filed: Mar. 22, 2024
(86) PCT No.: PCT/US2024/021130

§ 371 Date: Sep. 22, 2025

Related U.S. Application Data
(60) Provisional application No. 63/491,660, filed on Mar. 22, 2023.

(51) Int. Cl. C12N 15/90; C12N 9/22; C12N 15/11

(52) U.S. Cl. CPC C12N 15/907; C12N 9/226; C12N 15/11; C12N 2310/20

(57) ABSTRACT

RNA molecules comprising a guide sequence portion having 17-50 contiguous nucleotides in the sequence set forth in any one of SEQ ID NOs: 1-24195 and compositions, methods, and uses thereof.

This application claims the benefit of U.S. Provisional Patent Application No. 63/491,660, filed Mar. 22, 2023, the contents of which are hereby incorporated by reference into the application. Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “230322_92134-PRO_Sequence_Listing_AWG.xml”, which is 20,924 kilobytes in size, and which was created on Mar. 22, 2023 in the IBM-PC machine format, having an operating system compatibility with MS-Windows, which is contained in the XML file filed Mar. 22, 2024 as part of this application.

### BACKGROUND OF INVENTION

The Adeno-associated virus integration site 1 (AAVS1) safe harbor site may be targeted to introduce a desired sequence to the AAVS1 site without causing deleterious disruptions to the protein phosphatase 1, regulatory (inhibitor) subunit 12C (PPP1R12C) gene, which comprises the AAVS1 site, or affecting PPPTR12C expression or that of other endogenous genes. Such targeting strategies may be utilized to enable expression of an introduced sequence.

### SUMMARY OF THE INVENTION

Disclosed herein are strategies to enable the expression of a gene, or portion thereof, under control of endogenous promoters located in or near the AAVS1 locus, without interfering with the expression of endogenous genes or transcripts, including but not limited to PPP1R12C.

Also disclosed herein are strategies to enable the expression of an introduced gene or coding sequence, or portion thereof, independently of the endogenous promoters located in or near a targeted AAVS1 locus.

The present disclosure also provides a method for modifying in a cell at least one Adeno-associated virus integration site 1 (AAVS1) site, the method comprising
introducing to the cell a composition comprising:
   at least one CRISPR nuclease or a sequence encoding a CRISPR nuclease; and
an RNA molecule comprising a guide sequence portion having 17-50 nucleotides or a nucleotide sequence encoding the same,
wherein a complex of the CRISPR nuclease and the RNA molecule affects a double strand break in at the least one AAVS1 site.

In some embodiments, the composition also comprises a donor molecule, wherein a sequence of nucleotides from the donor molecule is inserted or copied at or near the double strand break site. Similarly, in some embodiments, the composition further comprises a donor molecule containing a sequence of nucleotides that is introduced at the double strand break site such that the expression of the introduced sequence is mediated by the promoter of an endogenous gene, or more preferably by an exogenously introduced promoter. For example, an exogenous promoter may be provided by the donor molecule.

According to embodiments of the present invention, there is provided a first RNA molecule comprising a guide sequence portion having 17-50 contiguous nucleotides containing nucleotides in the sequence set forth in any one of SEQ ID NOs: 1-24195. In some embodiments, the composition further comprises a CRISPR nuclease. In some embodiments, the composition further comprises a donor molecule. In some embodiments, the donor molecule is a single-stranded or double-stranded DNA molecule, plasmid. PCR product, adeno-associated virus (AVV), or intergrase deficient lentivirus. In some embodiments, the donor molecule is an RNA molecule.

In some embodiments, the donor consists of transcriptional elements that enable the independent expression of the inserted gene. Such elements can be but not limited to enhancers, core promoter elements, 5′UTR, 3′UTR and introns.

According to embodiments of the present invention, there is provided a cell modified by any one of the methods described herein. In some embodiments, the cell is a dividing cell. In some embodiments, the cell is a non-dividing cell. In some embodiments, the cell is a post-mitotic cell. In some embodiments, the cell is a stem cell. In some embodiments, the cell is a hematopoietic stem cell (HSC). In some embodiments, the cell is an induced pluripotent stem cell (iPSC). In some embodiments, the cell is ahematopoietic stem and progenitor cell (HSPC). In some embodiments, the cell is a lymphocyte. In some embodiments, the cell is a hepatocyte. In some embodiments, the cell is a neuron.

In some embodiments, the delivering of any one of the compositions described herein to the cell is performed in vitro, ex vivo, or in vivo. In some embodiments, the method is performed ex vivo and the cell is provided/explanted from an individual patient. In some embodiments, the method further comprises the step of introducing the resulting cell, with a modified or edited AAVS1 allele, into the individual patient (e.g. autologous transplantation).

According to some embodiments of the present invention, there is provided use of a composition comprising an RNA molecule comprising a guide sequence portion having 17-50 contiguous nucleotides containing nucleotides in the sequence set forth in any one of SEQ ID NOs: 1-24195 and a CRISPR nuclease for modifying or editing a AAVS1 allele in a cell, comprising delivering to the cell the composition comprising an RNA molecule comprising a guide sequence portion having 17-50 contiguous nucleotides containing nucleotides in the sequence set forth in any one of SEQ ID NOs: 1-24195 and a CRISPR nuclease. In some embodiments, the composition further comprises a donor molecule.

According to embodiments of the present invention, there is provided a medicament comprising an RNA molecule comprising a guide sequence portion having 17-50 contiguous nucleotides containing nucleotides in the sequence set forth in any one of SEQ ID NOs: 1-24195 and a CRISPR nuclease for use in modifying or editing a AAVS1 allele in a cell, wherein the medicament is administered by delivering to the cell the composition comprising an RNA molecule comprising a guide sequence portion having 17-50 contiguous nucleotides containing nucleotides in the sequence set forth in any one of SEQ ID NOs: 1-24195 and a CRISPR nuclease. In some embodiments, the medicament further comprises a donor molecule.

According to some embodiments of the present invention, there is provided a kit for modifying or editing AAVS1 allele in a cell, comprising an RNA molecule comprising a guide sequence portion having 17-50 contiguous nucleotides containing nucleotides in the sequence set forth in any one of SEQ ID NOs: 1-24195, a CRISPR nuclease, and/or a tracrRNA molecule; and instructions for delivering the RNA molecule; CRISPR nuclease, and/or the tracrRNA to the cell. In some embodiments, the kit further comprises a donor molecule.

### BRIEF DESCRIPTION OF THE DRAWINGS

FIG. 1A-1F: Editing and excision in HeLa cells using DNA transfection. HeLa cells were transfected with plasmids encoding the indicated nuclease and guide sequences. A total of sixteen (16) nucleases and thirty-nine (39) guide sequences were screened for editing activity. Cells were harvested 72 h post DNA transfection, genomic DNA was extracted, and the region of the target guide was amplified and analyzed by NGS. The graphs represent the % of editing±STDV from two independent experiments.

FIG. 1A provides the % editing associated with AAVS_s29, AAVS_s30, AAVS_s31. AAVS_s37, and AAVS_s43 using OMNI-159.

FIG. 1B provides the % editing associated with AAVS_s4 through AAVS_s7 and AAVS_s10 through AAVS_s15 using OMNI-103.

FIG. 1C provides the % editing associated with AAVS_s33 through AAVS_s35 using OMNI-110.

FIG. 1D provides the % editing associated with AAVS_s22 through AAVS_s26 and AAVS_s39 through AAVS_s41 using OMNI-274.

FIG. 1E provides the % editing associated with AAVS_s17 through AAVS_s19 using OMNI-308.

FIG. 1F provides the % editing associated with: AAVS_s3 using OMNI-50; AAVS_s39 and AAVS_s40 using OMNI-75; AAVS_s41 using OMNI-93; AAVS_s32 using OMNI-127: AAVS_s27 and AAVS_s28 using OMNI-231; AAVS_s43 using OMNI-269; AAVS_s20, AAVS_s21, and AAVS_s39 through AAVS_s42 using OMNI-281; AAVS_s37 using OMNI-286; AAVS_s36 and AAVS_s38 using OMNI-291; AAVS_s36 through AAVS_s38 using OMNI-302; and AAVS_s16 using OMNI-366.

### DETAILED DESCRIPTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

The term “homology-directed repair” or “HDR” refers to a mechanism for repairing DNA damage in cells, for example, during repair of double-stranded and single-stranded breaks in DNA. HDR requires nucleotide sequence homology and uses a “nucleic acid template” (nucleic acid template or donor template used interchangeably herein) to repair the sequence where the double-stranded or single break occurred (e.g., DNA target sequence). This results in the transfer of genetic information from, for example, the nucleic acid template to the DNA target sequence. HDR may result in alteration of the DNA target sequence (e.g., insertion, deletion, mutation) if the nucleic acid template sequence differs from the DNA target sequence and part or all of the nucleic acid template polynucleotide or oligonucleotide is incorporated into the DNA target sequence. In some embodiments, an entire nucleic acid template polynucleotide, a portion of the nucleic acid template polynucleotide, or a copy of the nucleic acid template is integrated at the site of the DNA target sequence.

The terms “nucleic acid template” and “donor”, refer to a nucleotide sequence that is inserted or copied into a genome. The nucleic acid template comprises a nucleotide sequence, e.g., of one or more nucleotides, that will be added to or will template a change in the target nucleic acid or may be used to modify the target sequence. A nucleic acid template sequence may be of any length, for example between 2 and 10,000 nucleotides in length. A nucleic acid template may be a single stranded nucleic acid, a double stranded nucleic acid. In some embodiments, the nucleic acid template comprises a nucleotide sequence, e.g., of one or more nucleotides, that corresponds to wild type sequence of the target nucleic acid, e.g., of the target position. In some embodiments, the nucleic acid template comprises a nucleotide sequence, e.g., of one or more ribonucleotides, that corresponds to wild type sequence of the target nucleic acid, e.g., of the target position. In some embodiments, the nucleic acid template comprises modified nucleotides.

Insertion of an exogenous sequence (also called a “donor sequence,” donor template,” “donor molecule” or “donor”) can also be carried out. For example, a donor sequence can contain a non-homologous sequence flanked by two regions of homology to allow for efficient homology directed repair (HDR) at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest. A donor molecule may be any length, for example ranging from several bases e.g. 10-20 bases to multiple kilobases in length.

The donor polynucleotide can be DNA or RNA, single-stranded and/or double-stranded and can be introduced into a cell in linear or circular form. See, e.g., U.S. Publication Nos. 2010/0047805; 2011/0281361; 2011/0207221; and 2019/0330620. See also Anzalone et al. (2019). If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) and Nehls et al. (1996). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor sequence may be an oligonucleotide and be used for targeted alteration of an endogenous sequence. The oligonucleotide may be introduced to the cell on a vector, may be electroporated into the cell, or may be introduced via other methods known in the art. Donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

As used herein, the term “modified cells” refers to cells in which a double strand break is affected by a complex of an RNA molecule and the CRISPR nuclease as a result of hybridization with the target sequence, i.e. on-target hybridization. The term “modified cells” may further encompass cells in which an edit or modification, including the introduction of an exogenous sequence, was affected following the double strand break.

This invention provides a modified cell or cells obtained by use of any of the methods described herein. In an embodiment these modified cell or cells are capable of giving rise to progeny cells. In an embodiment these modified cell or cells are capable of giving rise to progeny cells after engraftment. As a non-limiting example, the modified cells may be hematopoietic stem cells (HSCs), or any cell suitable for an allogenic cell transplant or autologous cell transplant. As a non-limiting example, the modified cells may be stem cells.

This invention also provides a composition comprising these modified cells and a pharmaceutically acceptable carrier. Also provided is an in vitro or ex vivo method of preparing this, comprising mixing the cells with the pharmaceutically acceptable carrier.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of an RNA molecule that can form a complex with a CRISPR nuclease, either alone or in combination with other RNA molecules, with the targeting sequence serving as the targeting portion of the CRISPR complex. When the molecule having the targeting sequence is present contemporaneously with the CRISPR molecule, the RNA molecule, alone or in combination with an additional one or more RNA molecules (e.g. a tracrRNA molecule), is capable of targeting the CRISPR nuclease to the specific target sequence. As non-limiting example, a guide sequence portion of a CRISPR RNA molecule or single-guide RNA molecule may serve as a targeting molecule. Each possibility represents a separate embodiment. A targeting sequence can be custom designed to target any desired sequence.

The term “targets” as used herein, refers to preferentially hybridizing a targeting sequence of a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur.

It is understood that where an RNA molecule targets a sequence, a complex of the RNA molecule and a CRISPR nuclease molecule targets the sequence for nuclease activity.

The “guide sequence portion” of an RNA molecule refers to a nucleotide sequence that is capable of hybridizing to a specific target DNA sequence, e.g., the guide sequence portion has a nucleotide sequence which is partially or fully complementary to the DNA sequence being targeted along the length of the guide sequence portion. In some embodiments, the guide sequence portion is 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length, or approximately 17-50, 17-49, 17-48, 17-47, 17-46, 17-45, 17-44, 17-43, 17-42, 17-41, 17-40, 17-39, 17-38,17-37, 17-36, 17-35, 17-34, 17-33, 17-31, 17-30, 17-29, 17-28, 17-27, 17-26, 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. Preferably, the entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the RNA molecule having the guide sequence portion is present contemporaneously with the CRISPR molecule, alone or in combination with an additional one or more RNA molecules (e.g. a tracrRNA molecule), the RNA molecule is capable of targeting the CRISPR nuclease to the specific target DNA sequence. Accordingly, a CRISPR complex can be formed by direct binding of the RNA molecule having the guide sequence portion to a CRISPR nuclease or by binding of the RNA molecule having the guide sequence portion and an additional one or more RNA molecules to the CRISPR nuclease. Each possibility represents a separate embodiment. A guide sequence portion can be custom designed to target any desired sequence. Accordingly, a molecule comprising a “guide sequence portion” is a type of targeting molecule. In some embodiments, the guide sequence portion comprises a sequence that is the same as, or differs by no more than 1, 2, 3, 4, or 5 nucleotides from, a guide sequence portion described herein, e.g., a guide sequence set forth in any of SEQ ID NOs: 1-24195. Each possibility represents a separate embodiment. In some of these embodiments, the guide sequence portion comprises a sequence that is the same as a sequence set forth in any of SEQ ID NOs: 1-24195. Throughout this application, the terms “guide molecule,” “RNA guide molecule,” “guide RNA molecule,” and “gRNA molecule” are synonymous with a molecule comprising a guide sequence portion.

The term “non-discriminatory” as used herein refers to a guide sequence portion of an RNA molecule that targets a specific DNA sequence that is common in both alleles of a gene. For example, a non-discriminatory guide sequence portion is capable of targeting both alleles of a gene present in a cell.

In embodiments of the present invention, an RNA molecule comprises a guide sequence portion having 17-50 contiguous nucleotides containing nucleotides in the sequence set forth in any one of SEQ ID NOs: 1-24195. In some embodiments, the guide sequence portion comprises a sequence that is the same as or differs by no more than 1, 2, or 3 nucleotides from a sequence set forth in any of SEQ ID NOs: 1-24195.

The RNA molecule and or the guide sequence portion of the RNA molecule may contain modified nucleotides. Exemplary modifications to nucleotides or polynucleotides may be synthetic and encompass polynucleotides which contain nucleotides comprising bases other than the naturally occurring adenine, cytosine, thymine, uracil, or guanine bases. Modifications to polynucleotides include polynucleotides which contain synthetic, non-naturally occurring nucleosides e.g., locked nucleic acids. Modifications to polynucleotides may be utilized to increase or decrease stability of an RNA. An example of a modified polynucleotide is an mRNA containing 1-methyl pseudo-uridine. For examples of modified polynucleotides and their uses, see U.S. Pat. No. 8,278,036, PCT International Publication No. WO/2015/006747, and Weissman and Kariko (2015), hereby incorporated by reference.

As used herein, “contiguous nucleotides” set forth in a SEQ ID NO refers to nucleotides in a sequence of nucleotides in the order set forth in the SEQ ID NO without any intervening nucleotides.

In embodiments of the present invention, the guide sequence portion may be 25 nucleotides in length and contain 20-22 contiguous nucleotides in the sequence set forth in any one of SEQ ID NOs: 1-24195. In embodiments of the present invention, the guide sequence portion may be less than 22 nucleotides in length. For example, in embodiments of the present invention the guide sequence portion may be 17, 18, 19, 20, or 21 nucleotides in length. In such embodiments the guide sequence portion may consist of 17, 18, 19, 20, or 21 nucleotides, respectively, in the sequence of 17-22 contiguous nucleotides set forth in any one of SEQ ID NOs: 1-24195. For example, a guide sequence portion having 17 nucleotides in the sequence of 17 contiguous nucleotides set forth in SEQ ID NO: 24196 may consist of any one of the following nucleotide sequences (nucleotides excluded from the contiguous sequence are marked in strike-through):

(SEQ ID NO: 24196)
AAAAUGUACUUGGUUCC
17 nucleotide guide sequence 1:
(SEQ ID NO: 24197) [symbol]
AAAAAUGUACUUGGUUCC
17 nucleotide guide sequence 2:
(SEQ ID NO: 24198) [symbol]
AAAAAAUGUACUUGGUUC [symbol]
17 nucleotide guide sequence 3:
(SEQ ID NO: 24199) [symbol]
AAAAAAUGUACUUGGUU [symbol]
17 nucleotide guide sequence 4:
(SEQ ID NO: 24200)
AAAAAAAUGUACUUGGU

[symbol]

In embodiments of the present invention, the guide sequence portion may be greater than 20 nucleotides in length. For example, in embodiments of the present invention the guide sequence portion may be 21, 22, 23, 24, 25, 26, 27, 28, 29, or 30 nucleotides in length. In such embodiments the guide sequence portion comprises 17-50 nucleotides containing the sequence of 20, 21 or 22 contiguous nucleotides set forth in any one of SEQ ID NOs: 1-24195 and additional nucleotides fully complimentary to a nucleotide or sequence of nucleotides adjacent to the 3′ end of the target sequence, 5′ end of the target sequence, or both.

In embodiments of the present invention a CRISPR nuclease and an RNA molecule comprising a guide sequence portion form a CRISPR complex that binds to a target DNA sequence to effect cleavage of the target DNA sequence. CRISPR nucleases, e.g. Cpf1, may form a CRISPR complex comprising a CRISPR nuclease and RNA molecule without a further tracrRNA molecule. Alternatively. CRISPR nucleases. e.g. Cas9, may form a CRISPR complex between the CRISPR nuclease, an RNA molecule, and a tracrRNA molecule. A guide sequence portion, which comprises a nucleotide sequence that is capable of hybridizing to a specific target DNA sequence, and a sequence portion that participates in CRIPSR nuclease binding, e.g. a tracrRNA sequence portion, can be located on the same RNA molecule. Alternatively, a guide sequence portion may be located on one RNA molecule and a sequence portion that participates in CRIPSR nuclease binding, e.g. a tracrRNA portion, may located on a separate RNA molecule. A single RNA molecule comprising a guide sequence portion (e.g. a DNA-targeting RNA sequence) and at least one CRISPR protein-binding RNA sequence portion (e.g. a tracrRNA sequence portion), can form a complex with a CRISPR nuclease and serve as the DNA-targeting molecule. In some embodiments, a first RNA molecule comprising a DNA-targeting RNA portion, which includes a guide sequence portion, and a second RNA molecule comprising a CRISPR protein-binding RNA sequence interact by base pairing to form an RNA complex that targets the CRISPR nuclease to a DNA target site or, alternatively, are fused together to form an RNA molecule that complexes with the CRISPR nuclease and targets the CRISPR nuclease to a DNA target site.

In embodiments of the present invention, a RNA molecule comprising a guide sequence portion may further comprise the sequence of a tracrRNA molecule. Such embodiments may be designed as a synthetic fusion of the guide portion of the RNA molecule and the trans-activating crRNA (tracrRNA). (See Jinek et al., 2012). In such an embodiment, the RNA molecule is a single guide RNA (sgRNA) molecule. Embodiments of the present invention may also form CRISPR complexes utilizing a separate tracrRNA molecule and a separate RNA molecule comprising a guide sequence portion. In such embodiments the tracrRNA molecule may hybridize with the RNA molecule via basepairing and may be advantageous in certain applications of the invention described herein.

The term “tracr mate sequence” refers to a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity. Gene modification can be achieved using a nuclease, for example a CRISPR nuclease.

According to embodiments of the present invention, there is provided an RNA molecule comprising a guide sequence portion (e.g. a targeting sequence) comprising a nucleotide sequence that is fully or partially complementary to a target located in or near an at least one AAVS1 site. In some embodiments, the guide sequence portion of the RNA molecule consists of 16, 17, 18, 19, 20, 21, 22, 23, 24, 25, 26, or more than 26 nucleotides. In some embodiments the guide sequence portion is configured to target a CRISPR nuclease to a AAVS1 target site and induce a double-strand break or a single-strand break within 500, 400, 300, 200, 100, 50, 25, or 10 nucleotides of the AAVS1 target site. In some embodiments, the RNA molecule is a guide RNA molecule such as a crRNA molecule or a single-guide RNA molecule. In some embodiments, the guide sequence portion is complementary to a target sequence located from 30 base pairs upstream to 30 base pairs downstream of Intron 1 of a PPP1R12C allele comprising a AAVS1 site. In some embodiments, the guide sequence portion is complementary to a target sequence located from 50 base pairs upstream to 50 base pairs downstream of Intron 1 of a PPP1R12C allele comprising a AAVS1 site. Each possibility represents a separate embodiment. In some embodiments, the guide sequence portion is complementary to a target sequence located from 7 base pairs upstream to 7 base pairs downstream of Intron 1 of a PPP1R12C allele comprising a AAVS1 site.

As used herein, the term “HSC” refers to both hematopoietic stem cells and hematopoietic stem progenitor cells. Non-limiting examples of stem cells include bone marrow cells, myeloid progenitor cells, a multipotent progenitor cells, and lineage restricted progenitor cells.

As used herein. “progenitor cell” refers to a lineage cell that is derived from stem cell and retains mitotic capacity and multipotency (e.g., can differentiate or develop into more than one but not all types of mature lineage of cell). As used herein “hematopoiesis” or “hemopoiesis” refers to the formation and development of various types of blood cells (e.g., red blood cells, megakaryocytes, myeloid cells (e.g., monocytes, macrophages and neutrophil), and lymphocytes) and other formed elements in the body (e.g., in the bone marrow).

According to some embodiments of the present invention, there is provided a method for modifying in a cell at least one Adeno-associated virus integration site 1 (AAVS1) site, the method comprising
introducing to the cell a composition comprising:
   at least one CRISPR nuclease or a sequence encoding a CRISPR nuclease; and
   a RNA molecule comprising a guide sequence portion having 17-50 nucleotides or a nucleotide sequence encoding the same,
wherein a complex of the CRISPR nuclease and the RNA molecule affects a double strand break in the at least one AAVS1 site.

In some embodiments, the composition also comprises a donor molecule. In some embodiments, a sequence of nucleotides from the donor molecule is inserted or copied at or near the double strand break site.

In some embodiments, the composition further comprises a donor molecule containing a sequence of nucleotides that is introduced at the double strand break site such that the expression of the introduced sequence is mediated by an endogenous promoter.

Alternatively, the donor molecule may contain a sequence of nucleotides that is introduced at the double strand break site which includes a promoter to mediate the expression of the introduced sequence.

In some embodiments, the introduced sequence is a sequence from an Alpha-1 antitrypsin, Glucose-6-phosphatase (G6PC), Serpin Family A Member (SERPINA), Transthyretin (TTR), ornithine transcarbamylase, argininosuccinic acid synthetase, arginase, argininosuccinase, carbamoyl phosphate synthetase, and N-acetylglutamate synthetase, Alpha Galactosidase A, Coagulation Factor IX, Coagulation Factor VII. Lysosomal Alpha Glucosidase, Fibrinogen, Phenylalanine 4 Hydroxylase, Alkaline Phosphatase, Glucosylceramidase, Beta Galactosidase, Porphobilinogen Deaminase, Arylsulfatase B, Beta Glucuronidase, Alpha-N-Acetylglucosaminidase, Lysosomal Alpha, Alpha L-Iduronidase, Mannosidase, Phosphatidylcholine Sterol Acyltransferase, N-Sulphoglucosamine Sulphohydrolase, Coagulation Factor X, N-Acetvlgalactosamine-6-Sulfatase, Sphingomyelin Phosphodiesterase, iduronate-2-sulfatase, Lysosomal Alpha Glucosidase, Cyclin Dependent Kinase Like 5, Prolow Density Lipoprotein Receptor Related Protein 1, Phenylalanine Ammonia Lyase. Protein Glutamine Gamma Glutamyltransferase K, or Lysosomal Protective Protein encoding gene.

In some embodiments, the introduced sequence is a sequence from an acid α-glucosidase, α-L-iduronidase, α-galactosidase, iduronate-2-sulfatase, N-acetylgalactosamine-6-sulfatase. N-acetylgalactosamine-4-sulfatase, a lysophosphatidylcholine metabolism-related protein, preferably phospholipase A2, a T-REC or K-REC related protein, β-glucosidase, β-glucocerebrosidase, arylsulfatase A, Factor VIII, insulin-like growth factor 1 (IGF-1), surfactant protein A, surfactant protein B, aspartyl-β-glucosaminidase, acetyl-CoA α-glucosaminide, acetyl-CoA-arylamine N-acetyltransferase, N-acetylglucosamine-6-sulfatase, N-acetylglucosamine-1-Phosphotransferase, α-N-acetylglucosaminidase, acid ceramidase, aspartacylase, lysosomal acid lipase, acid sphingomyelinase, arylsulfatase B, α-L-fucosidase, galactosylceramidase, galactocerebrosidase. β-galactosidase, protective protein/cathepsin A, β-glucoronidase, heparan N-sulfatase, β-hexosaminidase A, hyaluronidase-1, alpha-D-mannosidase, beta-mannosidase, alpha-neuraminidase, beta-hexosaminidase A, beta-hexosaminidase B, palmitoyl-protein thioesterase, tripeptidyl peptidase I, Battenin, Ceroid-lipofuscinosis neuronal protein 5 (CLN5), Ceroid-lipofuscinosis neuronal protein 6 (CLN6), Ceroid-lipofuscinosis neuronal protein 7 (CLN7), Ceroid-lipofuscinosis neuronal protein 8 (CLN8), (Cathepsin D), cystinosin, cathepsin K, Sialin, Lysosome-associated membrane protein 2 (LAMP2), human growth hormone, follicle-stimulating hormone, erythropoietin, or a granulocyte colony-stimulating factor (G-CSF) encoding gene.

In some embodiments, the donor molecule contains a sequence from a gene encoding a protein that is secreted by the cell.

In some embodiments, the RNA molecule comprises a non-discriminatory guide portion that targets both AAVS1 alleles.

In some embodiments, the RNA molecule comprises a non-discriminatory guide portion that targets any one of a non-discriminatory guide portion that targets Intron 1 of a PPP1R12C allele comprising a AAVS1 site.

In some embodiments, the RNA molecule comprises a non-discriminatory guide portion that targets a sequence that is located within a genomic range selected from any one of 19:55115657-55115880, 19:55115981-55117130, 19:55115881-55115980, 19:55115557-55115656, and 19:55112832-55115556.

According to embodiments of the present invention, there is provided a modified cell obtained by the method of any one of the embodiments presented herein.

According to some embodiments, the cell is a stem cell, a hematopoietic stem cell (HSC), an iPSC, a lymphocyte, a hepatocyte, or a neuron.

According to embodiments of the present invention, there is provided a RNA molecule comprising a guide sequence portion having 17-50 contiguous nucleotides containing nucleotides in the sequence set forth in any one of SEQ ID NOs: 1-24195.

According to embodiments of the present invention, there is provided a composition comprising the RNA molecule and at least one CRISPR nuclease.

According to some embodiments, the composition further comprises a donor molecule.

In some embodiments, the donor molecule contains a sequence from an Alpha-1 antitrypsin, Glucose-6-phosphatase (G6PC), Serpin Family A Member (SERPINA), Transthyretin (TTR), ornithine transcarbamylase, argininosuccinic acid synthetase, arginase, argininosuccinase, carbamoyl phosphate synthetase, and N-acetylglutamate synthetase, Alpha Galactosidase A, Coagulation Factor IX, Coagulation Factor VII, Lysosomal Alpha Glucosidase, Fibrinogen, Phenylalanine 4 Hydroxylase, Alkaline Phosphatase, Glucosylceramidase, Beta Galactosidase, Porphobilinogen Deaminase, Arylsulfatase B, Beta Glucuronidase, Alpha-N-Acetylglucosaminidase, Lysosomal Alpha, Alpha L-Iduronidase, Mannosidase, Phosphatidylcholine Sterol Acyltransferase, N-Sulphoglucosamine Sulphohydrolase, Coagulation Factor X, N-Acetylgalactosamine-6-Sulfatase, Sphingomyelin Phosphodiesterase, iduronate-2-sulfatase, Lysosomal Alpha Glucosidase, Cyclin Dependent Kinase Like 5, Prolow Density Lipoprotein Receptor Related Protein 1, Phenylalanine Ammonia Lyase. Protein Glutamine Gamma Glutamyltransferase K, or Lysosomal Protective Protein encoding gene.

In some embodiments, the donor molecule contains a sequence from an acid α-glucosidase, α-L-iduronidase, α-galactosidase, iduronate-2-sulfatase. N-acetylgalactosamine-6-sulfatase. N-acetylgalactosamine-4-sulfatase, a lysophosphatidylcholine metabolism-related protein, preferably phospholipase A2, a T-REC or K-REC related protein, β-glucosidase, β-glucocerebrosidase, arylsulfatase A, Factor VIII, insulin-like growth factor 1 (IGF-1), surfactant protein A, surfactant protein B, aspartyl-β-glucosaminidase, acetyl-CoA α-glucosaminide, acetyl-CoA-arylamine N-acetyltransferase. N-acetylglucosamine-6-sulfatase, N-acetylglucosamine-1-Phosphotransferase, α-N-acetylglucosaminidase, acid ceramidase, aspartacylase, lysosomal acid lipase, acid sphingomyelinase, arylsulfatase B, α-L-fucosidase, galactosylceramidase, galactocerebrosidase, β-galactosidase, protective protein/cathepsin A, β-glucoronidase, heparan N-sulfatase, β-hexosaminidase A, hyaluronidase-1, alpha-D-mannosidase, beta-mannosidase, alpha-neuraminidase, beta-hexosaminidase A, beta-hexosaminidase B, palmitoyl-protein thioesterase, tripeptidyl peptidase I, Battenin, Ceroid-lipofuscinosis neuronal protein 5 (CLN5), Ceroid-lipofuscinosis neuronal protein 6 (CLN6), Ceroid-lipofuscinosis neuronal protein 7 (CLN7), Ceroid-lipofuscinosis neuronal protein 8 (CLN8), (Cathepsin D), cystinosin, cathepsin K, Sialin, Lysosome-associated membrane protein 2 (LAMP2), human growth hormone, follicle-stimulating hormone, erythropoietin, or a granulocyte colony-stimulating factor (G-CSF) encoding gene.

In some embodiments, the donor molecule contains a sequence from a gene encoding a protein that is secreted by a cell.

According to some embodiments, the composition further comprises a tracrRNA molecule. According to embodiments of the present invention, there is provided a method for modifying or editing a AAVS1 allele in a cell, the method comprising delivering to the cell the composition of any one of the embodiments presented herein.

According to embodiments of the present invention, there is provided use of any one of the compositions presented herein for modifying or editing a AAVS1 allele in a cell, comprising delivering to the cell the composition of any one of the embodiments presented herein.

According to embodiments of the present invention, there is provided a medicament comprising the composition of any one of the embodiments presented herein for use in modifying or editing a AAVS1 allele in a cell, wherein the medicament is administered by delivering to the cell the composition of any one of the embodiments presented herein.

According to embodiments of the present invention, there is provided a kit for modifying or editing a AAVS1 allele in a cell, comprising an RNA molecule of any one of the embodiments presented herein, a CRISPR nuclease, and/or a tracrRNA molecule; and instructions for delivering the RNA molecule; CRISPR nuclease, and/or the tracrRNA to the cell. In some embodiments, the kit further comprises a donor molecule and instructions for delivering the donor molecule to a cell.

According to embodiments of the present invention, there is provided a gene editing composition comprising an RNA molecule comprising a guide sequence portion having 17-50 contiguous nucleotides containing nucleotides in the sequence set forth in any one of SEQ ID NOs: 1-24195. In some embodiments, the RNA molecule further comprises a portion having a sequence which binds to a CRISPR nuclease. In some embodiments, the sequence which binds to a CRISPR nuclease is a tracrRNA sequence.

In some embodiments, the RNA molecule further comprises a portion having a tracr mate sequence.

In some embodiments, the RNA molecule may further comprise one or more linker portions.

According to embodiments of the present invention, an RNA molecule may be up to 1000, 900, 800, 700, 600, 500, 450, 400, 350, 300, 290, 280, 270, 260, 250, 240, 230, 220, 210, 200, 190, 180, 170, 160, 150, 140, 130, 120, 110, or 100 nucleotides in length. Each possibility represents a separate embodiment. In embodiments of the present invention, the RNA molecule may be 17 up to 300 nucleotides in length, 100 up to 300 nucleotides in length, 150 up to 300 nucleotides in length, 100 up to 500 nucleotides in length, 100 up to 400 nucleotides in length, 200 up to 300 nucleotides in length. 100 to 200 nucleotides in length, or 150 up to 250 nucleotides in length. Each possibility represents a separate embodiment.

According to some embodiments of the present invention, the composition further comprises a tracrRNA molecule.

According to some embodiments of the present invention, there is provided a method for modifying or editing a AAVS1 allele in a cell, the method comprising delivering to the cell a composition comprising an RNA molecule comprising a guide sequence portion having 17-50 contiguous nucleotides containing nucleotides in the sequence set forth in any one of SEQ ID NOs: 1-24195 and a CRISPR nuclease. In some embodiments, the composition further comprises a donor molecule.

According to some embodiments of the present invention, there is provided a method for treating a disorder or disease, the method comprising delivering to a cell of a subject having the disorder or disease a composition comprising an RNA molecule comprising a guide sequence portion having 17-50 contiguous nucleotides containing nucleotides in the sequence set forth in any one of SEQ ID NOs: 1-24195 and a CRISPR nuclease. In some embodiments, the composition further comprises a donor molecule.

According to some embodiments of the present invention, there is provided a method for treating a disorder or disease, the method comprising delivering to a cell of a subject having the disorder the composition of any one of the above embodiments or delivering to the subject the modified cell of any one of the above embodiments.

According to some embodiments, the disease or disorder is Pompe disease, Mucopolysaccharidosis Type I. Fabry disease. Mucopolysaccharidosis type II, Mucopolysaccharidosis type IVA, Mucopolysaccharidosis type VI, Adrenoleukodystrophy, Severe combined immunodeficiency, Gaucher disease, metachromatic leukodystrophy (MLD), primary immune deficiency, hemophilia A, hemophilia B, IGF-1 deficiency, surfactant deficiency. Aspartylglycosaminuria, Sanfilipo syndrome, mucopolysaccharidosis type III, Sanfilippo Syndrome type IIId, 1-cell disease, Schindler Disease, Farber disease (FD), Spinal muscular atrophy with progressive myoclonic epilepsy (SMA-PME), Canavan disease, Lysosomal acid lipase deficiency, Niemann-Pick disease, Mucopolysaccharidosis type 6, Fucosidosis, Krabbe disease, GM1 gangliosidosis, mucopolysaccharidosis type IVB (MPS IVB), or galactosialidosis, Sly disease, Mucopolysaccharidosis type III, Late-onset Tay Sachs, Hyaluronidase-1 deficiency, Alpha-mannosidosis, Beta-mannosidosis, Sialidosis, Stanhoff disease, Santavuori-Haltia disease, Jansky-Bielschowsky disease, Batten disease, Neuronal ceroid lipofuscinosis type 5. Neuronal ceroid lipofuscinosis type 6, Neuronal ceroid lipofuscinosis type 7, Neuronal ceroid lipofuscinosis type 8, Congenital cathepsin D deficiency, Cystinosis, Pycnodysostosis, Salla disease, Danon disease, and/or Alpha-1 antitrypsin deficiency.

According to some embodiments of the present invention, there is provided a medicament comprising the composition any one of the above embodiments for use in modifying a AAVS1 allele in a cell, wherein the medicament is administered by delivering to the cell the composition any one of the above embodiments.

According to some embodiments of the present invention, there is provided use of the composition any one of the above embodiments or the modified cell of any one of the above embodiments for treating ameliorating or preventing a disorder or disease, comprising delivering to a cell of a subject having or at risk of having the disorder the composition any one of the above embodiments or delivering to the subject the modified cell of any one of the above embodiments.

According to some embodiments of the present invention, there is provided a medicament comprising the composition of any one of the above embodiments or the modified cell of any one of the above embodiments for use in treating ameliorating or preventing a disorder or disease, wherein the medicament is administered by delivering to a cell of a subject having or at risk of having the disorder the composition of any one of the above embodiments or delivering to the subject the modified cell of any one of the above embodiments.

According to some embodiments, the disorder or disease is Pompe disease, Mucopolysaccharidosis Type I, Fabry disease, Mucopolysaccharidosis type II, Mucopolysaccharidosis type IVA, Mucopolysaccharidosis type VI, Adrenoleukodystrophy, Severe combined immunodeficiency, Gaucher disease, metachromatic leukodystrophy (MLD), primary immune deficiency, hemophilia A, hemophilia B, IGF-1 deficiency, surfactant deficiency, Aspartylglycosaminuria, Sanfilipo syndrome, mucopolysaccharidosis type III, Sanfilippo Syndrome type IIId, I-cell disease, Schindler Disease, Farber disease (FD), Spinal muscular atrophy with progressive myoclonic epilepsy (SMA-PME), Canavan disease, Lysosomal acid lipase deficiency, Niemann-Pick disease, Mucopolysaccharidosis type 6, Fucosidosis, Krabbe disease, GM1 gangliosidosis, mucopolysaccharidosis type IVB (MPS IVB), or galactosialidosis, Sly disease, Mucopolysaccharidosis type III, Late-onset Tay Sachs, Hyaluronidase-1 deficiency, Alpha-mannosidosis, Beta-mannosidosis, Sialidosis, Stanhoff disease, Santavuori-Haltia disease, Jansky-Bielschowsky disease. Batten disease, Neuronal ceroid lipofuscinosis type 5, Neuronal ceroid lipofuscinosis type 6. Neuronal ceroid lipofuscinosis type 7, Neuronal ceroid lipofuscinosis type 8, Congenital cathepsin D deficiency, Cystinosis, Pycnodysostosis. Salla disease, Danon disease, and/or Alpha-1 antitrypsin deficiency.

In some embodiments, the disorder or disease is a lysosomal storage disorder.

In some embodiments, the disorder or disease is a disorder or disease of the blood, brain, lungs, or central nervous system.

In some embodiments, the composition of any one of the above embodiments or the modified cell of any one of the above embodiments is a medicament that is an enzyme replacement therapy.

According to some embodiments of the present invention, there is provided a method of treating a disease or disorder, wherein the method is an immunotherapy comprising delivering to the subject the modified cell of any one of the above embodiments.

In some embodiments, the disease or disorder is cancer.

In some embodiments, the composition of any one of the above embodiments or the modified cell of any one of the above embodiments is for use in treating ameliorating or preventing a disorder or disease.

According to some embodiments of the present invention, there is provided a method for modifying a DNA target site in a cell of a subject, wherein the modification of the DNA target site induces the cell to express a desired protein encoded by the modification, the method comprising delivering to the cell of the subject a composition comprising an RNA molecule comprising a guide sequence portion having 17-50 contiguous nucleotides containing nucleotides in the sequence set forth in any one of SEQ ID NOs: 1-24195 and a CRISPR nuclease. In some embodiments, the composition further comprises a donor molecule.

According to embodiments of the present invention, at least one CRISPR nuclease and the RNA molecule or RNA molecules are delivered to the subject and/or cells substantially at the same time or at different times.

In some embodiments, a tracrRNA molecule is delivered to the subject and/or cells substantially at the same time or at different times as the CRISPR nuclease and RNA molecule or RNA molecules.

According to embodiments of the present invention, the RNA molecule targets an alternative splicing signal sequence between an exon and an intron of a PPP1R12C allele comprising a AAVS1 site.

According to embodiments of the present invention, the RNA molecule is non-discriminatory and targets a sequence present in both PPP1R12C alleles. In some embodiments, the targeted sequence is present in both PPP1R12C alleles. In some embodiments, the sequence is present in an intron of the PPP1R12C gene.

Any one of, or combination of, the above-mentioned strategies for modifying or editing a an AAVS1 site may be used in the context of the invention.

In some embodiments, the method comprises contacting at least one allele of a gene of interest with anon-discriminatory RNA molecule, e.g. an RNA molecule comprising a guide sequence portion which is capable of targeting both alleles of a gene, and a CRISPR nuclease e.g., a Cas9 protein, wherein the non-discriminatory RNA molecule and the CRISPR nuclease associate with a nucleotide sequence of the at least one allele of the gene of interest, thereby modifying or editing the at least one allele. Notably, although biallelic cleavage may occur upon introduction of a non-discriminatory RNA molecule to a cell, insertion of a nucleotide sequence at a cleavage site may occur in only a single allele and not in both alleles. Accordingly, inducing biallelic cleavage with a non-discriminatory RNA molecule that targets an intron of PPP1R12C allele comprising a AAVS1 site may result in preservation of expression of an endogenous PPP1R12C gene from one allele and introduction of a nucleotide sequence, e.g. a nucleotide sequence from a donor molecule, in the other PPP1R12C allele. Introduction of the nucleotide sequence in a AAVS1 site may or may not disrupt expression of the PPP1R12C encoded gene product comprising the AAVS1 site.

In some embodiments, the method comprises contacting an allele of a gene of interest with an RNA molecule and a CRISPR nuclease e.g., a Cas9 protein, wherein the RNA molecule and the CRISPR nuclease associate with a nucleotide sequence of the allele of the gene of interest which differs by at least one nucleotide from a nucleotide sequence of a different allele of the gene of interest, thereby modifying or editing the targeted allele.

In some embodiments, the RNA molecule and a CRISPR nuclease is introduced to a cell encoding the gene of interest. In some embodiments, the cell encoding the gene of interest is in a mammalian subject.

Embodiments of compositions described herein include at least one CRISPR nuclease, RNA molecule(s) comprising a guide sequence portion, and a tracrRNA molecule, which may be separate or attached to an RNA molecule comprising a guide sequence portion, being effective in a subject or cells at the same time. The at least one CRISPR nuclease, RNA molecule(s) comprising a guide sequence portion, and tracrRNA may be delivered substantially at the same time or can be delivered at different times but have effect at the same time. For example, this includes delivering the CRISPR nuclease to the subject or cells before the RNA molecule comprising a guide sequence portion and/or tracrRNA is substantially extant in the subject or cells.

In some embodiments, the cell is a dividing cell. In some embodiments, the cell is a non-dividing cell. In some embodiments, the cell is a post-mitotic cell. In some embodiments, the cell is a stem cell. In some embodiments, the cell is a hematopoietic stem cell (HSC). In some embodiments, the cell is an induced pluripotent stem cell (iPSC). In some embodiments, the cell is a hematopoietic stem and progenitor cell (HSPC). In some embodiments, the cell is a lymphocyte. In some embodiments, the cell is a hepatocyte. In some embodiments, the cell is a neuron.

### Genetic AAVS1 Safe Harbor Knock-Ins to Treat Diseases and Disorders

In some embodiments, methods of the present invention may be used to knock-in a sequence at a AAVS1 safe harbor site. In some embodiments, AAVS1-mediated expression of the knocked-in sequence is involved in or associated with treatment of a disorder or a disease.

For example, in some embodiments a AAVS1 DNA target site in target cell is modified such that the target cell expresses and secretes a protein product encoded by the modification, e.g. an introduced or knocked-in protein-encoding sequence. These target cells may be utilized, for example, to treat lysosomal storage diseases or other disorders of the blood, lungs, brain, or central nervous system. In some embodiments, these modified cells serve as an alternative to traditional enzyme replacement therapies. In some embodiments, these modified cells are used for immunotherapy such as cancer immunotherapy.

As a non-limiting example, expression of the knocked-in sequence may be involved in or associated with treatment of a disease or disorder of the blood, lungs, brain, or central nervous system. As non-limiting examples, the knocked in sequence may be a A1AT, G6PC, SERPINA, TTR, omithine transcarbamylase, argininosuccinic acid synthetase, arginase, argininosuccinase, carbamoyl phosphate synthetase, or N-acetylglutamate synthetase sequence, or a portion thereof.

As a non-limiting example, expression of the knocked-in sequence may be involved in or associated with treatment of a lysosomal storage disease or other disorder. As a non-limiting example, a target cell (e.g. a monocyte or macrophage) may be modified to express Alpha Galactosidase A, Coagulation Factor IX, Coagulation Factor VII, Lysosomal Alpha Glucosidase, Fibrinogen, Phenylalanine 4 Hydroxylase, Alkaline Phosphatase, Glucosylceramidase. Beta Galactosidase, Porphobilinogen Deaminase. Arylsulfatase B. Beta Glucuronidase, Alpha-N-Acetylglucosaminidase, Lysosomal Alpha. Alpha L-Iduronidase, Mannosidase, Phosphatidylcholine Sterol Acyltransferase, N-Sulphoglucosamine Sulphohydrolase, Coagulation Factor X, N-Acetylgalactosamine-6-Sulfatase, Sphingomyelin Phosphodiesterase, Alpha-1 antitrypsin, iduronate-2-sulfatase, Lysosomal Alpha Glucosidase, Cyclin Dependent Kinase Like 5, Prolow Density Lipoprotein Receptor Related Protein 1, Phenylalanine Ammonia Lyase, Protein Glutamine Gamma Glutamyltransferase K, Lysosomal Protective Protein, or a portion thereof.

As non-limiting examples, expression of the knocked-in sequence may be involved in or associated with treatment of any one of the following diseases or disorders (which are each followed by a related gene or enzyme in parentheses): Pompe disease (acid α-glucosidase). Mucopolysaccharidosis Type I (α-L-iduronidase), Fabry disease (α-galactosidase), Mucopolysaccharidosis type II (iduronate-2-sulfatase), Mucopolysaccharidosis type IVA (N-acetylgalactosamine-6-sulfatase), Mucopolysaccharidosis type VI (N-acetylgalactosamine-4-sulfatase), Adrenoleukodystrophy (a lysophosphatidylcholine metabolism-related gene, e.g. phospholipase A2), Severe combined immunodeficiency (T-REC or K-REC related gene), Gaucher disease (β-glucosidase or β-glucocerebrosidase), metachromatic leukodystrophy (MLD) (arylsulfatase A), primary immune deficiency, hemophilia A and B (Factor VIII), IGF-1 deficiency (IGF-1), surfactant deficiency (surfactant protein A (SP-A) and/or surfactant protein B (SP-B), Aspartylglycosaminuria (aspartyl-R-glucosaminidase), Sanfilipo syndrome (acetyl-CoA α-glucosaminide), mucopolysaccharidosis type III (acetyl-CoA-arylamine N-acetyltransferase), Sanfilippo Syndrome type IId (N-acetylglucosamine-6-sulfatase), I-cell disease (N-acetylglucosamine-1-Phosphotransferase), Schindler Disease (α-N-acetylglucosaminidase), Farber disease (FD) or Spinal muscular atrophy with progressive mvoclonic epilepsy (SMA-PME) (acid ceramidase), Canavan disease (aspartacylase), Lysosomal acid lipase deficiency (lysosomal acid lipase), Niemann-Pick disease (acid sphingomyelinase), Mucopolysaccharidosis type 6 (arylsulfatase B), Fucosidosis (α-L-fucosidase), Krabbe disease (galactosylceramidase, galactocerebrosidase), GM1 gangliosidosis, mucopolysaccharidosis type IVB (MPS IVB), or galactosialidosis (galactasidase-beta-1 or β-galactosidase (GLB1), or protective protein/cathepsin A), Sly disease (β-glucoronidase), Mucopolysaccharidosis type III (heparan N-sulfatase), Late-onset Tay Sachs (β-hexosaminidase A), Hyaluronidase-1 deficiency (hyaluronidase-1), Alpha-mannosidosis (alpha-D-mannosidase), Beta-mannosidosis (beta-mannosidase), Sialidosis (alpha-neuraminidase). Stanhoff disease (beta-hexosaminidase A and/or beta-hexosaminidase B), Santavuori-Haltia disease (palmitoyl-protein thioesterase), Jansky-Bielschowsky disease (tripeptidyl peptidase I), Batten disease (Battenin), Neuronal ceroid lipofuscinosis type 5 (Ceroid-lipofuscinosis neuronal protein 5 (CLN5)), Neuronal ceroid lipofuscinosis type 6 (CLN6), Neuronal ceroid lipofuscinosis type 7 (CLN7), Neuronal ceroid lipofuscinosis type 8 (CLN8), Congenital cathepsin D deficiency (Cathepsin D), Cystinosis (cystinosin), Pycnodysostosis (cathepsin K), Salla disease (Sialin), and/or Danon disease (Lysosome-associated membrane protein 2 (LAMP2)). As a non-limiting example, a target cell (e.g. a monocyte or macrophage) may be modified to express a metabolic modulator. As a non-limiting example, a target cell may be modified to express human growth hormone, insulin-like growth factor 1 (IGF-1). Factor VIII (hemophilia A and B), follicle-stimulating hormone, erythropoietin, granulocyte colony-stimulating factor (G-CSF), galactosamine-6-sulfatase, and/or β-hexosamini enzymes.

Specifically, cells which reside in target tissues, including in the lungs and brain, and can serve as an expression vectors for long-term secretion of proteins in those target tissues. Expression of a transgene under the control of a AAVS1 promoter is achieved by CRISPR mediated knock-in at a safe harbor site that is targeted by the guide sequence portions described herein. The protein product of the expressed transgene may be secreted. Accordingly, the ability to generate blood and/or tissues containing modified target cells which continuously secrete a protein of interest serves as an alternative to enzyme replacement therapy (ERT). For example, modified monocytes may be useful to target the central nervous system (CNS) or the lungs and secrete a protein of interest in the target tissue.

Such an approach is also useful, for example, to treat lysosomal storage diseases. Some of these diseases also display a phenotype in the CNS. Notably, there is a challenge to treat brain damage with enzyme replacement therapy due to the brain blood barrier (BBB). Monocytes may be utilized for delivery across the BBB such that the secreted protein of interest will be secreted in the CNS.

For example, modified cells delivered to the brain may be utilized to treat a lysosomal storage disease; modified cells delivered to the lungs may be utilized to treat anti-trypsin deficiency (A1AT), D-Surfactant deficiency, or proteinosis; or modified cells in the blood may be utilized to treat AAT or deficiency of adenosine deaminase 2 (DADA2).

### CRISPR Nucleases and PAM Recognition

In some embodiments, the sequence specific nuclease is selected from CRISPR nucleases, or a functional variant thereof. In some embodiments, the sequence specific nuclease is an RNA-guided DNA nuclease. In some embodiments, the CRISPR complex does not further comprise a tracrRNA. In a non-limiting example, in which the RNA-guided DNA nuclease is a CRISPR protein, the at least one nucleotide which differs between AAVS1 alleles may be within the PAM site and/or proximal to the PAM site within the region that the RNA molecule is designed to hybridize to. A skilled artisan will appreciate that RNA molecules can be engineered to bind to a target of choice in a genome by commonly known methods in the art.

The term “PAM” as used herein refers to a nucleotide sequence of a target DNA located in proximity to the targeted DNA sequence and recognized by the CRISPR nuclease complex. The PAM sequence may differ depending on the nuclease identity. In addition, there are CRISPR nucleases that can target almost all PAMs. In some embodiments of the present invention, a CRISPR system utilizes one or more RNA molecules having a guide sequence portion to direct a CRISPR nuclease to a target DNA site via Watson-Crick base-pairing between the guide sequence portion and the protospacer on the target DNA site, which is next to the protospacer adjacent motif (PAM), which is an additional requirement for target recognition. The CRISPR nuclease then mediates cleavage of the target DNA site to create a double-stranded break within the protospacer. In a non-limiting example, a type II CRISPR system utilizes a mature crRNA:tracrRNA complex that directs the CRISPR nuclease, e.g. Cas9 to the target DNA the target DNA via Watson-Crick base-pairing between the guide sequence portion of the crRNA and the protospacer on the target DNA next to the protospacer adjacent motif (PAM). A skilled artisan will appreciate that each of the engineered RNA molecule of the present invention is further designed such as to associate with a target genomic DNA sequence of interest next to a protospacer adjacent motif (PAM), e.g., a PAM matching the sequence relevant for the type of CRISPR nuclease utilized, such as for a non-limiting example, NGG or NAG, wherein “N” is any nucleobase, for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant; NGCG for SpCas9-VRER variant; NGAG for SpCas9-EQR variant; NRRH for SpCas9-NRRH variant, wherein N is any nucleobase, R is A or G and H is A, C, or T; NRTH for SpCas9-NRTH variant, wherein N is any nucleobase, R is A or G and H is A, C, or T; NRCH for SpCas9-NRCH variant, wherein N is any nucleobase, R is A or G and H is A, C, or T; NG for SpG variant of SpCas9 wherein N is any nucleobase; NG or NA for SpCas9-NG variant of SpCas9 wherein N is any nucleobase: NR or NRN or NYN for SpRY variant of SpCas9, wherein N is any nucleobase, R is A or G and Y is C or T; NNG for Streptococcus canis Cas9 variant (ScCas9), wherein N is any nucleobase: NNNRRT for SaKKH-Cas9 variant of Staphylococcus aureus (SaCas9), wherein N is any nucleobase, and R is A or G; NNNNGATT for Neisseria meningitidis (NmCas9), wherein N is any nucleobase; TTN for Alicyclobacillus acidiphilus Cas12b (AacCas12b), wherein N is any nucleobase; or TTTV for Cpf1, wherein V is A. C or G. RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments, an RNA-guided DNA nuclease e.g., a CRISPR nuclease, may be used to cause a DNA break, either double or single-stranded in nature, at a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Publication No. 2015/0211023, incorporated herein by reference.

CRISPR systems that may be used in the practice of the invention vary greatly. CRISPR systems can be a type I, a type II, or a type III system. Non-limiting examples of suitable CRISPR proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cul966.

In some embodiments, the RNA-guided DNA nuclease is a CRISPR nuclease derived from a type II CRISPR system (e.g., Cas9). The CRISPR nuclease may be derived from Streptococcus pyogenes, Streptococcus thermophilus, Streptococcus sp., Staphylococcus aureus, Neisseria meningitidis, Treponema denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difjicile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, or any species which encodes a CRISPR nuclease with a known PAM sequence. CRISPR nucleases encoded by uncultured bacteria may also be used in the context of the invention. (See Burstein et al. Nature, 2017). Variants of CRIPSR proteins having known PAM sequences e.g., SpCas9 D1135E variant, SpCas9 VQR variant. SpCas9 EQR variant, or SpCas9 VRER variant may also be used in the context of the invention.

Thus, an RNA-guided DNA nuclease of a CRISPR system, such as a Cas9 protein or modified Cas9 or homolog or ortholog of Cas9, or other RNA-guided DNA nucleases belonging to other types of CRISPR systems, such as Cpf1 and its homologs and orthologs, may be used in the compositions of the present invention. Additional CRISPR nucleases may also be used, for example, the nucleases described in PCT International Application Publication Nos. WO2020/223514, WO2020/223553, WO2022/087135, WO2022/170199, WO2022/170216, WO2022/226215, WO2023/091987, WO2023/019269, which are hereby incorporated by reference.

In certain embodiments, the CRIPSR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Derivatives include, but are not limited to, CRISPR nickases, catalytically inactive or “dead” CRISPR nucleases, and fusion of a CRISPR nuclease or derivative thereof to other enzymes such as base editors or retrotransposons. See for example, Anzalone et al. (2019) and PCT International Application No. PCT/US2020/037560.

Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

In some embodiments, the CRISPR nuclease is Cpf1. Cpf1 is a single RNA-guided endonuclease which utilizes a T-rich protospacer-adjacent motif. Cpf1 cleaves DNA via a staggered DNA double-stranded break. Two Cpf1 enzymes from Acidaminococcus and Lachnospiraceae have been shown to carry out efficient genome-editing activity in human cells. (See Zetsche et al., 2015).

Thus, an RNA-guided DNA nuclease of a Type II CRISPR System, such as a Cas9 protein or modified Cas9 or homologs, orthologues, or variants of Cas9, or other RNA-guided DNA nucleases belonging to other types of CRISPR systems, such as Cpf1 and its homologs, orthologues, or variants, may be used in the present invention.

In some embodiments, the guide molecule comprises one or more chemical modifications which imparts a new or improved property (e.g., improved stability from degradation, improved hybridization energetics, or improved binding properties with an RNA-guided DNA nuclease). Suitable chemical modifications include, but are not limited to: modified bases, modified sugar moieties, or modified inter-nucleoside linkages. Non-limiting examples of suitable chemical modifications include: 4-acetylcytidine, 5-(carboxyhydroxymethyl)uridine. 2′-O-methylcytidine, 5-carboxymethylaminomethyl-2-thiouridine, 5-carboxymethylaminomethyluridine, dihydrouridine, 2′-O-methylpseudouridine, “beta, D-galactosylqueuosine”, 2′-O-methylguanosine, inosine, N6-isopentenyladenosine, 1-methyladenosine, 1-methylpseudouridine, 1-methylguanosine, 1-methylinosine, “2,2-dimethylguanosine”, 2-methyladenosine, 2-methylguanosine, 3-methylcytidine, 5-methylcytidine, N6-methyladenosine. 7-methylguanosine, 5-methylaminomethyluridine. 5-methoxyaminomethyl-2-thiouridine, “beta, D-mannosylqueuosine”, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl)threonine, uridine-5-oxyacetic acid-methylester, uridine-5-oxyacetic acid, wybutoxosine, queuosine, 2-thiocytidine, 5-methyl-2-thiouridine, 2-thiouridine, 4-thiouridine, 5-methyluridine, N-((9-beta-D-ribofuranosylpurine-6-yl)-carbamoyl)threonine, 2′-O-methyl-5-methyluridine, 2′-O-methyluridine, wybutosine, “3-(3-amino-3-carboxy-propyl)uridine, (acp3)u”. 2′-O-methyl (M). 3′-phosphorothioate (MS), 3′-thioPACE (MSP), pseudouridine, or 1-methyl pseudo-uridine. Each possibility represents a separate embodiment of the present invention.

Guide Sequences which Target a AAVS1 Allele

Any given RNA molecule comprising a guide sequence portion utilized to target a DNA site may result in degradation of the RNA molecule, limited activity, no activity, or off-target effects. Accordingly, suitable guide sequence portions are necessary for targeting a given DNA site in a gene.

By the present invention, a novel set of guide sequence portions have been identified for targeting at least one AAVS1 allele and introducing to the at least one allele a sequence of nucleotides to be expressed under the control of the AAVS1 promoter. Such a gene editing approach may be used to treat a disorder or disease or modify behavior of a cell. Preferably, a non-discriminatory RNA molecule capable of targeting both AAVS1 alleles is used for targeting.

In some embodiments of the present invention, an RNA molecule is used to target a AAVS1 site to introduce, or knock-in, an exogenous sequence of nucleotides into the AAVS1 site. In some embodiments, the location of the target site is near the intended knock-in site, preferably near a start codon or stop codon, preferably within 150 nucleotides of a start codon or stop codon.

### Delivery to Cells

The compositions described herein may be delivered to a target cell by any suitable means. Compositions of the present invention may be targeted to any cell which contains and/or expresses a AAVS1 allele, including any mammalian cell. For example, in one embodiment an RNA molecule that specifically targets a AAVS1 allele is delivered to a target cell and the target cell is a stem cell, a hematopoietic stem cell (HSC), an iPSC, a lymphocyte, a hepatocyte, or a neuron. The delivery to the cell may be performed in vitro, ex vivo, or in vivo. Further, the nucleic acid compositions described herein may be delivered as one or more of DNA molecules, RNA molecules, ribonucleoproteins (RNPs), nucleic acid vectors, or any combination thereof.

In some embodiments, in vivo delivery methods of the compositions described herein include delivery by a lentivirus, adeno-associated virus (AAV) or nanoparticle. In some embodiments, in vivo delivery methods of the compositions described herein include delivery by a lentivirus, adeno-associated virus (AAV) or nanoparticle. The composition may be in the form of an RNP composition. Accordingly, the delivery can be in vivo to cells within a subject.

In some embodiments, any one of the compositions described herein is delivered to a cell ex vivo. In some embodiments, the cell is a dividing cell. In some embodiments, the cell is a non-dividing cell. In some embodiments, the cell is a post-mitotic cell. In some embodiments, the cell is a stem cell. In some embodiments, the cell is a hematopoietic stem cell (HSC). In some embodiments, the cell is an induced pluripotent stem cell (iPSC). In some embodiments, the cell is a hematopoietic stem and progenitor cell (HSPC). In some embodiments, the cell is a lymphocyte. In some embodiments, the cell is a hepatocyte. In some embodiments, the cell is a neuron. The composition may be delivered to the cell by any known ex vivo delivery method, including but not limited to, electroporation, viral transduction, nanoparticle delivery, liposomes, etc. The composition may be in the form of an RNP composition. Additional detailed delivery methods are described throughout this section.

In some embodiments, an RNA molecule of a composition described herein comprises a chemical modification. Non-limiting examples of suitable chemical modifications include 2′-0-methyl (M), 2-0-methyl, 3′phosphorothioate (MS) or 2′-0-methyl, 3′thioPACE (MSP), pseudouridine, and 1-methyl pseudo-uridine. Each possibility represents a separate embodiment of the present invention.

Any suitable viral vector system may be used to deliver nucleic acid compositions e.g., the RNA molecule compositions of the subject invention. Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson (1992); Nabel & Felgner (1993); Mitani & Caskey (1993); Dillon (1993); Miller (1992); Van Brunt (1988); Vigne (1995): Kremer & Perricaudet (1995); Haddada et al. (1995); and Yu et al. (1994).

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus). (See, e.g., Chung et al., 2006). Sonoporation using, e.g., the Sonitron 2000 system (Rich-Mar), can also be used for delivery of nucleic acids. Cationic-lipid mediated delivery of proteins and/or nucleic acids is also contemplated as an in vivo, ex vivo, or in vitro delivery method. (See Zuris et al. (2015); see also Coelho et al. (2013): Judge et al. (2006); and Basha et al. (2011)).

Non-viral vectors, such as transposon-based systems e.g. recombinant Sleeping Beauty transposon systems or recombinant PiggyBac transposon systems, may also be delivered to a target cell and utilized for transposition of a polynucleotide sequence of a molecule of the composition or a polynucleotide sequence encoding a molecule of the composition in the target cell.

Additional exemplary nucleic acid delivery systems include those provided by Amaxa.® Biosystems (Cologne, Germany), Maxcyte, Inc. (Rockville, Md.), BTX Molecular Delivery Systems (Holliston, Mass.) and Copernicus Therapeutics Inc., (see. e.g., U.S. Pat. No. 6,008,336). Lipofection is described in e.g., U.S. Pat. Nos. 5,049,386, 4,946,787; and U.S. Pat. No. 4,897,355, and lipofection reagents are sold commercially (e.g., Transfectam™, Lipofectin™ and Lipofectamine™. RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995): Blaese et al., (1995); Behr et al., (1994); Remy et al. (1994): Gao and Huang (1995); Ahmad and Allen (1992); U.S. Pat. Nos. 4,186,183: 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (See MacDiarmid et al., 2009).

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (See, e.g., Buchschacher et al. (1992); Johann et al. (1992); Sommerfelt et al. (1990); Wilson et al. (1989); Miller et al. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (See Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors. (Ellem et al., (1997); Dranoff et al., 1997).

Packaging cells are used to form virus particles that are capable of infecting a host cell. Such cells include 293 cells, which package adenovirus, AAV, and Psi-2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravitreal, intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, optionally after selection for cells which have incorporated the vector. A non-limiting exemplary ex vivo approach may involve removal of tissue (e.g., peripheral blood, bone marrow, and spleen) from a patient for culture, nucleic acid transfer to the cultured cells (e.g., hematopoietic stem cells), followed by grafting the cells to a target tissue (e.g., bone marrow, and spleen) of the patient. In some embodiments, the stem cell or hematopoietic stem cell may be further treated with a viability enhancer.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid composition, and re-infused back into the subject organism (e.g., patient). Various cell types suitable for ex vivo transfection are well known to those of skill in the art (See, e.g., Freshney, “Culture of Animal Cells, A Manual of Basic Technique and Specialized Applications (6th edition, 2010) and the references cited therein for a discussion of how to isolate and culture cells from patients).

Suitable cells include, but are not limited to, eukaryotic cells and/or cell lines. Non-limiting examples of such cells or cell lines generated from such cells include COS, CHO (e.g., CHO-S, CHO-KI, CHO-DG44, CHO-DUXB11, CHO-DUKX, CHOKISV), VERO, MDCK, WI38, V79, B14AF28-G3, BHK, HaK, NSO, SP2/0-Agl4, HeLa, HEK293 (e.g., HEK293-F, HEK293-H, HEK293-T), perC6 cells, any plant cell (differentiated or undifferentiated), as well as insect cells such as Spodopterafugiperda (Sf), or fungal cells such as Saccharomyces, Pichia and Schizosaccharomvces. In certain embodiments, the cell line is a CHO-K1, MDCK or HEK293 cell line. Additionally, primary cells may be isolated and used ex vivo for reintroduction into the subject to be treated following treatment with a guided nuclease system (e.g. CRISPR/Cas). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-gamma, and TNF-alpha are known (as a non-limiting example see, Inaba et al., 1992).

Stem cells are isolated for transduction and differentiation using known methods. For example, stem cells are isolated from bone marrow cells by panning the bone marrow cells with antibodies which bind unwanted cells, such as CD4+ and CD8+(T cells), CD45+ (panB cells), GR-1 (granulocytes), and Lad (differentiated antigen presenting cells) (as a non-limiting example, see Inaba et al., 1992). Stem cells that have been modified may also be used in some embodiments.

Vectors (e.g., retroviruses, liposomes, etc.) containing therapeutic nucleic acid compositions can also be administered directly to an organism for transduction of cells in vivo. Administration is by any of the routes normally used for introducing a molecule into ultimate contact with blood or tissue cells including, but not limited to, injection, infusion, topical application (e.g., eye drops and cream) and electroporation. Suitable methods of administering such nucleic acids are available and well known to those of skill in the art, and, although more than one route can be used to administer a particular composition, a particular route can often provide a more immediate and more effective reaction than another route. According to some embodiments, the composition is delivered via IV injection.

Vectors suitable for introduction of transgenes into immune cells (e.g., T-cells) include non-integrating lentivirus vectors. See, e.g., U.S. Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (See. e.g., Remington's Pharmaceutical Sciences, 17th ed., 1989).

The disclosed compositions and methods may also be used in the manufacture of a medicament for treating a disease or disorder in a patient.

### AAVS1 Safe Harbor Knock-In Methods

Without being bound by any theory or mechanism, the instant invention may be utilized to apply a CRISPR nuclease to process a AAVS1 site, also referred to as a AAVS1 locus, in order to introduce a sequence to a safe harbor site within the AAVS1 locus. A specific guide sequence may be selected from Table 1 based on the targeted position and the type of CRISPR nuclease used (e.g. according to a required PAM sequence).

The goal of the presented strategies is to knock-in an expression cassette within an AAVS1 safe harbor site following a CRISPR-mediated DNA break. A coding sequence introduced into a AAVS1 safe harbor site may be expressed under the control of an endogenous promoter. More preferably, an introduced coding sequence may be expressed via transcription elements supplied with an exogenous donor molecule. For example, an HDR cassette may contain both a sequence of interest and a promoter to control the expression of the sequence of interest, such that both the sequence of interest and the promoter are introduced into a AAVS1 safe harbor site following a CRISPR-mediated DNA break.

For example, strategies to induce breaks and knock-in an expression cassette via homology directed repair (HDR) or homology-independent targeted insertion (HITI) include, but are not limited to (1) targeting a PPP1R12C allele comprising a AAVS1 site with a nuclease and one RNA guide molecule to mediate a double-strand break; and (2) targeting an a PPP1R12C allele comprising a AAVS1 site with two single-strand nickases and two guide RNA molecules (one guide for each nickase), thus directing the nickases to mediate nicks on the targeted, complimentary DNA strands and thereby forming a double strand break.

Furthermore, a donor molecule may be used to introduce a desired sequence of nucleotides into a AAVS1 safe harbor site via knock-in. The donor molecule comprising a template of the insert sequence may be, for example, a single stranded DNA, plasmid, PCR product, AAV, or intergrase-deficient lentivirus. Also, the insert sequence may be introduced as an RNA template in CRISPR-based systems, including but not limited to reverse transcriptase editors, directed by at least one guide RNA molecule.

All dividing and non-dividing cells, such as but not restricted to hematopoietic stem cells, iPSCs, lymphocytes, hepatocytes, neurons.

### Examples of RNA Guide Sequences which Specifically Target a AAVS1 Site

Although a large number of guide sequences can be designed to target a AAVS1 allele, the nucleotide sequences described in Table 1 identified by SEQ ID NOs: 1-24195 below were specifically selected to effectively implement the methods set forth herein and to effectively discriminate between alleles.

Table 1 lists guide sequences designed for use as described in the embodiments above to associate specific sequences within a AAVS1 allele. Each engineered guide molecule is further designed such as to associate with a target genomic DNA sequence of interest that lies next to a protospacer adjacent motif (PAM), e.g., a PAM matching the sequence NGG or NAG, where “N” is any nucleobase. The guide sequences were designed to work in conjunction with one or more different CRISPR nucleases, including, but not limited to, e.g. SpCas9WT (PAM SEQ: NGG), SpCas9.VQR.1 (PAM SEQ: NGAN), SpCas9.VQR.2 (PAM SEQ: NGNG), SpCas9.EQR (PAM SEQ: NGAG), SpCas9.VRER (PAM SEQ: NGCG), SaCas9WT (PAM SEQ: NNGRRT), SpRY (PAM SEQ: NRN or NYN), NmCas9WT (PAM SEQ: NNNNGATT), Cpf1 (PAM SEQ: TTTV), or JeCas9WT (PAM SEQ: NNNVRYM). RNA molecules of the present invention are each designed to form complexes in conjunction with one or more different CRISPR nucleases and designed to target polynucleotide sequences of interest utilizing one or more different PAM sequences respective to the CRISPR nuclease utilized.

TABLE 1
Guide sequence portions designed to associate with specific AAVSI site targets

| | SEQ ID NOS: | SEQ ID NOS: | SEQ ID NOS: |
|---|---|---|---|
| | of 20 base | of 21 base | of 22 base |
| Target | guides | guides | guides |
| 100 bp Upstream and downstream | 1-394 | 395-786 | 787-1176 |
| from insertion site | | | |
| chr19:55115657-55115880 | | | |
| Downstream edge of AAVS site | 1177-3418 | 3419-5660 | 5661-7902 |
| chr19:55115981-55117130 | | | |
| Extra 100 bp downstream from cut | 7903-8048 | 8049-8192 | 8193-8334 |
| site | | | |
| chr19:55115881-55115980 | | | |
| Extra 100 bp upstream from cut site | 8335-8480 | 8481-8624 | 8625-8766 |
| chr19:55115557-55115656 | | | |
| Upstream edge of AAVS site | 8767-13890 | 13891-19034 | 19035-24195 |
| chr19:55112832-55115556 | | | |

The indicated locations listed in column 1 of the Table 1 are based on gnomAD v3 database and UCSC Genome Browser assembly ID: hg38, Sequencing/Assembly provider ID: Genome Reference Consortium Human GRCh38.p12 (GCA_000001405.27). Assembly date: Dec. 2013 initial release; Dec. 2017 patch release 12.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### Experimental Details

### Example 1: AAVS1 On-Target Activity Analysis

Guide sequences comprising 17-50 contiguous nucleotides containing nucleotides in the sequence set forth in any one of SEQ ID NOs: 1-24195 are screened for high on-target activity using a CRISPR nuclease, e.g. OMNI-50, OMNI-79, or OMNI-103, in HeLa cells. On-target activity is determined by DNA capillary electrophoresis analysis. Description of the OMNI-50, OMNI-79, and OMNI-103 nucleases are provided in PCT International Application Publication Nos. WO 2020/223514 A2, WO 2021/248016 A2, and WO 2022/170199 A2, the contents of each of which are hereby incorporated by reference.

### Example 2: AAVS1 On-Target Activity Analysis

A total of sixteen (16) nucleases and thirty-nine (39) guide sequences were screened for editing activity. HeLa cells were transfected with plasmids encoding the indicated nuclease and guide sequences, as set forth in Table 2. Cells were harvested 72 h post DNA transfection, genomic DNA was extracted, and the region of the target guide was amplified and analyzed by NGS. Table 2 includes the average % of editing and standard deviation from two independent experiments.

TABLE 2
% Editing using indicated guide sequences and nucleases

| | | | SEQ. | | | |
|---|---|---|---|---|---|---|
| CRISPR | | | ID | | | |
| Nuclease | Guide Name | Guide Sequence Portion | NO | PAM | Average % Edits | STDEV |
| OMNI-50 | AAVS_s3 | TGTCCCTAGTGGCCCCACTGTG | 1157 | GGGTGGAG | 12.22666667 | 0.729817329 |
| OMNI-75 | AAVS_s39 | ACCAGAGCCACATTAACCGGCC | 814 | CTGGGAAT | 0.513333333 | 0.094516313 |
| | AAVS_s40 | CCAGGGATCCTGTGTCCCCGAG | 910 | CTGGGACC | 0.623333333 | 0.368962509 |
| OMNI-93 | AAVS_s41 | GGGAATATAAGGTGGTCCCAGC | 1042 | TCGGGGAC | 51.91 | 9.219985539 |
| OMNI-103 | AAVS_s4 | ACACTGGGTGGGCTCCGGGACC | 19276 | TGAACTGG | 15.27 | 3.435365095 |
| | AAVS_s5 | ACTTATTCTGATTTTGTTTTTC | 19494 | CAAACTGC | 0.13 | 0.02 |
| | AAVS_s6 | AGTCCTCCTTACCATCCCTCCC | 6031 | TCGACTTC | 41.10 | 3.965669342 |
| | AAVS_s7 | CAGAATAAGTTGGTCCTGAGTT | 20193 | CTAACTTT | 11.64 | 1.696201639 |
| | AAVS_s10 | CGCACGTGCCCTGGGAACGGGA | 21084 | TGAACTCG | 23.99 | 1.234274416 |
| | AAVS_s11 | GAGCCAAAGTTAGAACTCAGGA | 21802 | CCAACTTA | 20.33 | 5.191159151 |
| | AAVS_s12 | GGAGCTGAGGAAGGAGTGAAGC | 22373 | TAAACTCC | 32.67 | 4.367680544 |
| | AAVS_s13 | TCTCTTCCGCATTGGAGTCGCT | 7636 | TTAACTGG | 13.44 | 4.26704816 |
| | AAVS_s14 | TGCCTTTTGGGTGGATTCTCCT | 23722 | CCAACTGT | 87.21 | 2.560449179 |
| | AAVS_s15 | TTTCGTGGCTCCCTCTGGAGTC | 24160 | CCGACTTG | 10.08 | 1.177582269 |
| OMNI-110 | AAVS_s33 | AAGGATGGGGCTTTTCTGTCAC | 798 | CAATCCTG | 52.48 | 4.739581557 |
| | AAVS_s34 | CAGGATTGGTGACAGAAAAGCC | 892 | CCATCCTT | 74.09 | 8.624482593 |
| | AAVS_s35 | GTCCCCGAGCTGGGACCACCTT | 1074 | ATATTCCC | 0.15 | 0.101488916 |
| OMNI-127 | AAVS_s32 | GCCCTGGGAATATAAGGTGGTC | 1011 | CCAGCTCG | 2.343333333 | 0.303699413 |
| OMNI-159 | AAVS_s43 | CCCCGAGCTGGGACCACCTTAT | 926 | ATTCCCAG | 70.09666667 | 2.919474839 |
| | AAVS_s29 | GCCTAAGGATGGGGCTTTTCTG | 1014 | TCACCAAT | 70.6 | 2.867635495 |
| | AAVS_s37 | GGCCTAAGGATGGGGCTTTTCT | 1038 | GTCACCAA | 85.485 | 5.208006656 |
| | AAVS_s30 | GGGTACTTTTATCTGTCCCCTC | 1056 | CACCCCAC | 20.68666667 | 0.351188458 |
| | AAVS_s31 | TAGTCTCCTGATATTGGGTCTA | 1105 | ACCCCCAC | 76.1 | 1.250333289 |
| OMNI-231 | AAVS_s27 | GCCTCCAGGGATCCTGTGTCCC | 1015 | CGAGCTGG | 16.3 | 1.075546373 |
| | AAVS_s28 | TAGGGACAGGATTGGTGACAGA | 1104 | AAAGCCCC | 4.6 | 0.574543297 |
| OMNI-269 | AAVS_s43 | CCCCGAGCTGGGACCACCTTAT | 926 | ATTCCCAG | 0.66 | 0.445458565 |
| OMNI-274 | AAVS_s39 | ACCAGAGCCACATTAACCGGCC | 814 | CTGGGAAT | 0.07 | 0.078102497 |
| | AAVS_s40 | CCAGGGATCCTGTGTCCCCGAG | 910 | CTGGGACC | 0.03 | 4.65661E-10 |
| | AAVS_s22 | CCTCCACCCCACAGTGGGGCCA | 939 | CTAGGGAC | 0.11 | 0.02081666 |
| | AAVS_s23 | GAGCTGGGACCACCTTATATTC | 996 | CCAGGGCC | 0.18 | 0.170098011 |
| | AAVS_s41 | GGGAATATAAGGTGGTCCCAGC | 1042 | TCGGGGAC | 0.45 | 0.180092569 |
| | AAVS_s24 | GGTTAGACCCAATATCAGGAGA | 1068 | CTAGGAAG | 53.90 | 7.699820344 |
| | AAVS_s25 | TGGGTCTAACCCCCACCTCCTG | 1150 | TTAGGCAG | 26.31 | 2.32796764 |
| | AAVS_s26 | TGGTGACAGAAAAGCCCCATCC | 1152 | TTAGGCCT | 20.84 | 3.745882184 |
| OMNI-281 | AAVS_s39 | ACCAGAGCCACATTAACCGGCC | 814 | CTGGGAAT | 0.28 | 0.180277564 |
| | AAVS_s20 | CCAGCTCGGGGACACAGGATCC | 909 | CTGGAGGC | 4.14 | 1.597884017 |
| | AAVS_s40 | CCAGGGATCCTGTGTCCCCGAG | 910 | CTGGGACC | 0.04 | 0.011547005 |
| | AAVS_s21 | CCCAGAACCAGAGCCACATTAA | 917 | CCGGCCCT | 0.11 | 0.049328829 |
| | AAVS_s41 | GGGAATATAAGGTGGTCCCAGC | 1042 | TCGGGGAC | 0.76 | 0.195021366 |
| | AAVS_s42 | TGCCTCCAGGGATCCTGTGTCC | 1139 | CCGAGCTG | 1.11 | 0.361155553 |
| OMNI-286 | AAVS_s37 | GGCCTAAGGATGGGGCTTTTCT | 1038 | GTCACCAA | 1.14 | 0.833386665 |
| OMNI-291 | AAVS_s36 | AAGCCCCATCCTTAGGCCTCCT | 795 | CCTTCCTA | 8.87 | 2.689690193 |
| | AAVS_s38 | GGGGCTTTTCTGTCACCAATCC | 1052 | TGTCCCTA | 0.10 | 0.083864971 |
| OMNI-302 | AAVS_s36 | AAGCCCCATCCTTAGGCCTCCT | 795 | CCTTCCTA | 1.48 | 0.490034012 |
| | AAVS_s37 | GGCCTAAGGATGGGGCTTTTCT | 1038 | GTCACCAA | 2.79 | 1.251572344 |
| | AAVS_s38 | GGGGCTTTTCTGTCACCAATCC | 1052 | TGTCCCTA | 1.06 | 1.000616477 |
| OMNI-308 | AAVS_s17 | CTAACAGGAGGTGGGGGTTAGA | 953 | CCCAATAT | 28.61 | 4.579523265 |
| | AAVS_s18 | CTGTGTCCCCGAGCTGGGACCA | 977 | CCTTATAT | 0.07 | 0.072341781 |
| | AAVS_s19 | TAGGCCTCCTCCTTCCTAGTCT | 1103 | CCTGATAT | 8.83 | 1.674126638 |
| OMNI-366 | AAVS_s16 | TTCCTAGTCTCCTGATATTGGG | 1168 | TCTAACCC | 0.02 | 0.005773503 |

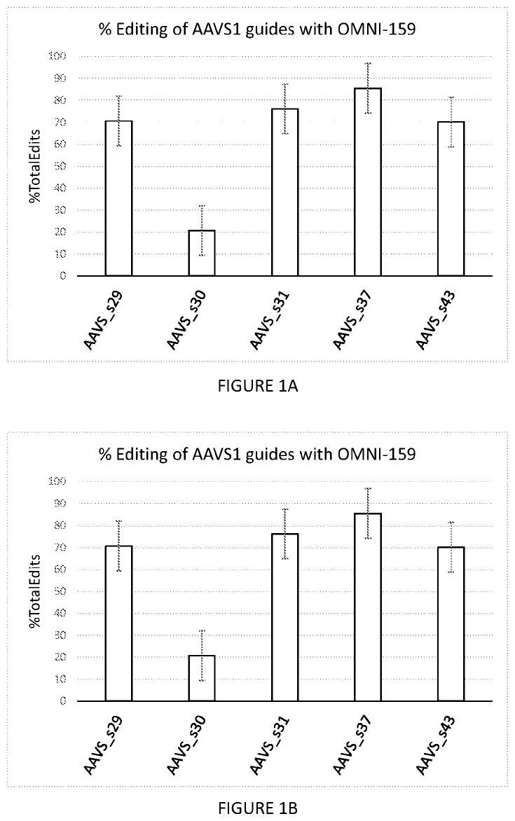

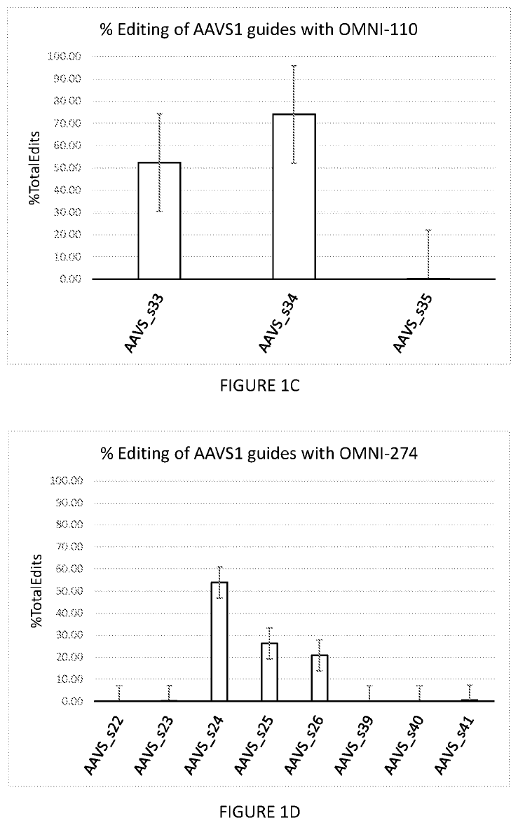

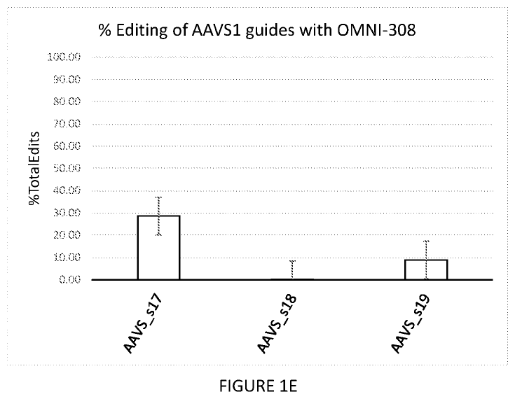

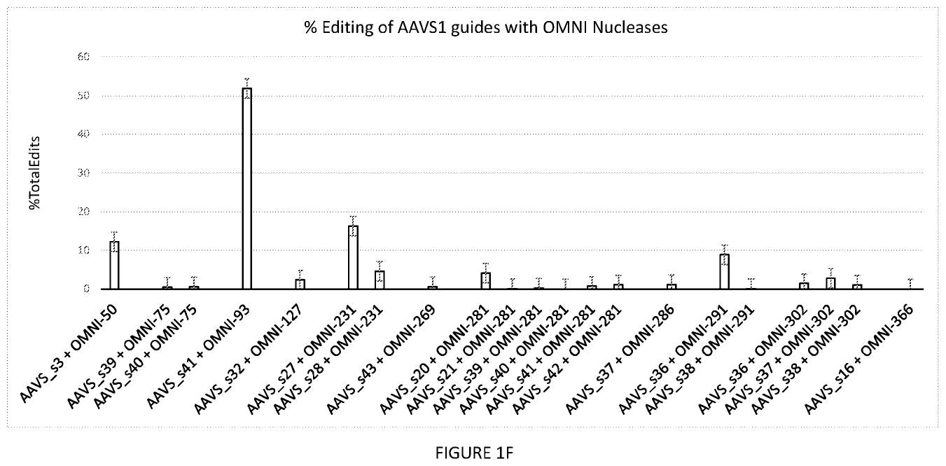

FIG. 1A-II provide the Table 2 data in graphical form, proving the % of editing for various OMNI nucleases by guide sequence.

FIG. 1A provides the % editing associated with AAVS_s29, AAVS_s30, AAVS_s31, AAVS_s37, and AAVS_s43 using OMNI-159.

FIG. 1B provides the % editing associated with AAVS_s4 through AAVS_s7 and AAVS_s10 through AAVS_s15 using OMNI-103.

FIG. 1C provides the % editing associated with AAVS_s33 through AAVS_s35 using OMNI-110.

FIG. 1D provides the % editing associated with AAVS_s22 through AAVS_s26 and AAVS_s39 through AAVS_s41 using OMNI-274.

FIG. 1E provides the % editing associated with AAVS_s17 through AAVS_s19 using OMNI-308.

FIG. 1F provides the % editing associated with: AAVS_s3 using OMNI-50; AAVS_s39 and AAVS_s40 using OMNI-75; AAVS_s41 using OMNI-93; AAVS_s32 using OMNI-127; AAVS_s27 and AAVS_s28 using OMNI-231: AAVS_s43 using OMNI-269; AAVS_s20, AAVS_s21, and AAVS_s39 through AAVS_s42 using OMNI-281; AAVS_s37 using OMNI-286; AAVS_s36 and AAVS_s38 using OMNI-291; AAVS_s36 through AAVS_s38 using OMNI-302; and AAVS_s16 using OMNI-366.

The description of the OMNI nucleases in Example 2 are described in PCT International Application Publication Nos. WO2020/223514 (OMNI-50); WO2022/087135 (OMNI-75); WO2022/170199 (OMNI-103): WO2022/170216 (OMNI-93, OMNI-110); WO2022/226215 (OMNI-231); WO2023/091987 (OMNI-269, OMNI-274, OMNI-281, OMNI-286, OMNI-291, OMNI-302, OMNI-308, OMNI-366); and WO2023/019269 (OMNI-127, OMNI-159), the contents of each of which are hereby incorporated by reference.

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20.
2. Anderson (1992) “Human gene therapy”, Science 256:808-13.
3. Anzalone et al. (2019) “Search-and-replace genomme editing without double-strand brakes or donor DNA”. Nature 576, 149-157.
4. Basha et al. (2011) “Influence of Cationic Lipid Composition on Gene Silencing Properties of Lipid Nanoparticle Formulations of siRNA in Antigen-Presenting Cells”, Mol. Ther. 19(12):2186-200.
5. Behr (1994) Gene transfer with synthetic cationic amphiphiles: Prospects for gene therapy”, Bioconjuage Chem 5:382-89.
6. Blaese (1995) “Vectors in cancer therapy: how will they deliver”, Cancer Gene Ther. 2:291-97.
7. Blaese et al. (1995) “T lympocyte-directed gene therapy for ADA-SCID: initial trial results after 4 years”, Science 270(5235):475-80.
8. Buchschacher and Panganiban (1992) “Human immunodeficiency virus vectors for inducible expression of foreign genes”, J. Virol. 66:2731-39.
9. Burstein et al. (2017) “New CRISPR-Cas systems from uncultivated microbes”, Nature 542:237-41.
10. Chang and Wilson (1987) “Modification of DNA ends can decrease end-joining relative to homologous recombination in mammalian cells”, Proc. Natl. Acad. Sci. USA 84:4959-4963.
11. Chung et al. (2006) “Agrobacterium is not alone: gene transfer to plants by viruses and other bacteria”, Trends Plant Sci. 11(1):1-4.
12. Coelho et al. (2013) “Safety and efficacy of RNAi therapy for transthyretin amyloidosis” N. Engl. J. Med. 369, 819-829.
13. Crystal (1995) “Transfer of genes to humans: early lessons and obstacles to success”, Science 270(5235):404-10.
14. Dillon (1993) “Regulation gene expression in gene therapy” Trends in Biotechnology 11(5):167-173.
15. Dranoff et al. (1997) “A phase I study of vaccination with autologous, irradiated melanoma cells engineered to secrete human granulocyte macrophage colony stimulating factor”, Hum. Gene Ther. 8(1):111-23.
16. Dunbar et al. (1995) “Retrovirally marked CD34-enriched peripheral blood and bone marrow cells contribute to long-term engraftment after autologous transplantation”, Blood 85:3048-57.
17. Ellem et al. (1997) “A case report: immune responses and clinical course of the first human use of ganulocyte/macrophage-colony-stimulating-factor-tranduced autologous melanoma cells for immunotherapy”, Cancer Immunol Immunother 44:10-20.
18. Gao and Huang (1995) “Cationic liposome-mediated gene transfer” Gene Ther. 2(10):710-22.
19. Haddada et al. (1995) “Gene Therapy Using Adenovirus Vectors”, in: The Molecular Repertoire of Adenoviruses III: Biology and Pathogenesis, ed. Doerfler and Bohm, pp. 297-306.
20. Han et al. (1995) “Ligand-directed retro-viral targeting of human breast cancer cells”, Proc Natl Acad Sci U.S.A. 92(21):9747-51.
21. Inaba et al. (1992) “Generation of large numbers of dendritic cells from mouse bone marrow cultures supplemented with granulocyte/macrophage colony-stimulating factor”. J Exp Med. 176(6):1693-702.
22. Jinek et al. (2012) “A programmable dual-RNA-guided DNA endonuclease in adaptive bacterial immunity,” Science 337(6096):816-21.
23. Johan et al. (1992) “GLVR1, a receptor for gibbon ape leukemia virus, is homologous to a phosphate permease of Neurospora crassa and is expressed at high levels in the brain and thymus”, J Virol 66(3):1635-40.
24. Judge et al. (2006) “Design of noninflammatory synthetic siRNA mediating potent gene silencing in vivo”, Mol Ther. 13(3):494-505.
25. Kohn et al. (1995) “Engraftment of gene-modified umbilical cord blood cells in neonates with adnosine deaminase deficiency”, Nature Medicine 1:1017-23.
26. Kremer and Perricaudet (1995) “Adenovirus and adeno-associated virus mediated gene transfer”, Br. Med. Bull. 51(1):31-44.
27. Macdiarmid et al. (2009) “Sequential treatment of drug-resistant tumors with targeted minicells containing siRNA or a cytotoxic drug”, Nat Biotehcnol. 27(7):643-51.
28. Malech et al. (1997) “Prolonged production of NADPH oxidase-corrected granulocyes after gene therapy of chronic granulomatous disease”, PNAS 94(22):12133-38.
29. Miller et al. (1991) “Construction and properties of retrovirus packaging cells based on gibbon ape leukemia virus”, J Virol. 65(5):2220-24.
30. Miller (1992) “Human gene therapy comes of age”, Nature 357:455-60.
31. Mitani and Caskey (1993) “Delivering therapeutic genes—matching approach and application”, Trends in Biotechnology 11(5):162-66.
32. Nabel and Felgner (1993) “Direct gene transfer for immunotherapy and immunization”, Trends in Biotechnology 11(5):211-15.
33. Remy et al. (1994) “Gene Transfer with a Series of Lipphilic DNA-Binding Molecules”, Bioconjugate Chem. 5(6):647-54.
34. Sentmanat et al. (2018) “A Survey of Validation Strategies for CRISPR-Cas9 Editing”, Scientific Reports 8:888. doi:10.1038/s41598-018-19441-8.
35. Sommerfelt et al. (1990) “Localization of the receptor gene for type D simian retroviruses on human chromosome 19”. J. Virol. 64(12):6214-20.
36. Van Brunt (1988) “Molecular framing: transgenic animals as bioactors” Biotechnology 6:1149-54.
37. Vigne et al. (1995) “Third-generation adenovectors for gene therapy”, Restorative Neurology and Neuroscience 8(1,2): 35-36.
38. Weissman and Kariko (2015) “mRNA:Fulfilling the promise of gene therapy”, Molecular Therapy (9):1416-7.
39. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
40. Yu et al. (1994) “Progress towards gene therapy for HIV infection”, Gene Ther. 1(1):13-26.
41. Zetsche et al. (2015) “Cpf1 is a single RNA-guided endonuclease of a class 2 CRIPSR-Cas system” Cell 163(3):759-71.
42. Zuris et al. (2015) “Cationic lipid-mediated delivery of proteins enables efficient protein based genome editing in vitro and in vivo” Nat Biotechnol. 33(1):73-80.

1. A method for modifying in a cell at least one Adeno-associated virus integration site 1 (AAVS1) site, the method comprising
introducing to the cell a composition comprising:
at least one CRISPR nuclease, or a DNA polynucleotide encoding the at least one CRISPR nuclease; and
an RNA molecule, or a DNA polynucleotide encoding the RNA molecule,
comprising a guide sequence portion having 17-50 nucleotides of the nucleotide sequence set forth in any one of SEQ ID NOs: 1-24195,
wherein the at least one CRISPR nuclease and the RNA molecule form a complex, and wherein the complex binds to at least one AAVS1 site.

2. The method of claim 1, wherein the composition further comprises a donor molecule containing a sequence of nucleotides that is introduced at the double strand break site such that the expression of the introduced sequence is mediated by the promoter of an endogenous gene, or more preferably by an exogenous promoter.

3. The method of claim 2, wherein the introduced sequence is a sequence from an Alpha-1 antitrypsin, Glucose-6-phosphatase (G6PC), Serpin Family A Member (SERPINA), Transthyretin (TTR), ornithine transcarbamylase, argininosuccinic acid synthetase, arginase, argininosuccinase, carbamoyl phosphate synthetase, and N-acetylglutamate synthetase, Alpha Galactosidase A, Coagulation Factor IX, Coagulation Factor VII, Lysosomal Alpha Glucosidase, Fibrinogen, Phenylalanine 4 Hydroxylase, Alkaline Phosphatase, Glucosylceramidase, Beta Galactosidase, Porphobilinogen Deaminase, Arylsulfatase B, Beta Glucuronidase, Alpha-N-Acetylglucosaminidase, Lysosomal Alpha, Alpha L-Iduronidase, Mannosidase, Phosphatidylcholine Sterol Acyltransferase, N-Sulphoglucosamine Sulphohydrolase, Coagulation Factor X, N-Acetylgalactosamine-6-Sulfatase, Sphingomyelin Phosphodiesterase, iduronate-2-sulfatase, Lysosomal Alpha Glucosidase, Cyclin Dependent Kinase Like 5, Prolow Density Lipoprotein Receptor Related Protein 1, Phenylalanine Ammonia Lyase, Protein Glutamine Gamma Glutamyltransferase K. or Lysosomal Protective Protein encoding gene; or wherein the introduced sequence is a sequence from an acid a-glucosidase, a-L-iduronidase, a-galactosidase, iduronate-2-sulfatase, N-acetylgalactosamine-6-sulfatase, N-acetylgalactosamine-4-sulfatase, a lysophosphatidylcholine metabolism-related protein, preferably phospholipase A2, a T-REC or K-REC related protein, p-glucosidase, P-glucocerebrosidase, arylsulfatase A, Factor VIII, insulin-like growth factor 1 (IGF-1), surfactant protein A, surfactant protein B, aspartyl-a-glucosaminidase, acetyl-CoA a-glucosaminide, acetyl-CoA-arylamine N-acetyltransferase, N-acetylglucosamine-6-sulfatase, N-acetylglucosamine-1-Phosphotransferase, a-N-acetylglucosaminidase, acid ceramidase, aspartacylase, lysosomal acid lipase, acid sphingomyelinase, arylsulfatase B, a-L-fucosidase, galactosylceramidase, galactocerebrosidase, P-galactosidase, protective protein/cathepsin A, a-glucoronidase, heparan N-sulfatase, P-hexosaminidase A, hyaluronidase-1, alpha-D-mannosidase, beta-mannosidase, alphaneuraminidase. beta-hexosaminidase A, beta-hexosaminidase B. palmitoyl-protein thioesterase, tripeptidyl peptidase I, Battenin, Ceroid-lipofuscinosis neuronal protein 5 (CLN5), Ceroid-lipofuscinosis neuronal protein 6 (CLN6), Ceroid-lipofuscinosis neuronal protein 7 (CLN7), Ceroid-lipofuscinosis neuronal protein 8 (CLN8), (Cathepsin D), cystinosin, cathepsin K, Sialin, Lysosome-associated membrane protein 2 (LAMP2), human growth hormone, follicle-stimulating hormone, erythropoietin, CD-19 or a granulocyte colony-stimulating factor (G-CSF) encoding gene.

4. (canceled)

5. (canceled)

6. The method of claim 1, wherein the RNA molecule comprises a non-discriminatory guide portion that targets a PPP1R12C allele comprising a AAVS1 site.

7. The method of claim 1, wherein the RNA molecule comprises a non-discriminatory guide portion that targets a sequence that is located within a genomic range selected from any one of 19:55115657-55115880, 19:55115981-55117130, 19:55115881-55115980. 19:55115557-55115656. and 19:55112832-55115556.

8. The method of claim 1, wherein a PPP1R12C allele comprising the at least AAVS1 site expresses a PPP1R12C gene product.

9. A modified cell obtained by the method of claim 1.

10. The modified cell of claim 9, wherein the modified cell is a stem cell, a hematopoietic stem cell (HSC), an iPSC, a lymphocyte, a hepatocyte, or a neuron.

11. An RNA molecule, or a DNA polynucleotide encoding the RNA molecule, that targets an AAVS1 allele, wherein RNA molecule comprises a guide sequence portion comprising 17-50 contiguous nucleotides of the nucleic acid sequence set forth in any one of SEQ ID NOs: 1-24195.

12. A composition of claim 11, further comprising at least one CRISPR nuclease.

13. The composition of claim 11, further comprising a donor molecule.

14. The composition of claim 13, wherein the donor molecule contains a sequence from an Alpha-1 antitrypsin, Glucose-6-phosphatase (G6PC), Serpin Family A Member (SERPINA), Transthyretin (TTR), ornithine transcarbamylase, argininosuccinic acid synthetase, arginase, argininosuccinase, carbamoyl phosphate synthetase, and N-acetylglutamate synthetase, Alpha Galactosidase A, Coagulation Factor IX, Coagulation Factor VIE Lysosomal Alpha Glucosidase, Fibrinogen, Phenylalanine 4 Hydroxylase. Alkaline Phosphatase. Glucosylceramidase, Beta Galactosidase, Porphobilinogen Deaminase, Arylsulfatase B, Beta Glucuronidase, Alpha-N-Acetylglucosaminidase, Lysosomal Alpha, Alpha L-Iduronidase, Mannosidase, Phosphatidylcholine Sterol Acyltransferase, N-Sulphoglucosamine Sulphohydrolase. Coagulation Factor X, N-Acetylgalactosamine-6-Sulfatase, Sphingomyelin Phosphodiesterase, iduronate-2-sulfatase. Lysosomal Alpha Glucosidase, Cyclin Dependent Kinase Like 5, Prolow Density Lipoprotein Receptor Related Protein 1, Phenylalanine Ammonia Lyase, Protein Glutamine Gamma Glutamyltransferase K. or Lysosomal Protective Protein encoding gene; or wherein the donor molecule contains a sequence from an acid a-glucosidase, a-L-iduronidase, a-galactosidase. iduronate-2-sulfatase, N-acetylgalactosamine-6-sulfatase, N-acetylgalactosamine-4-sulfatase, a lysophosphatidylcholine metabolism-related protein, preferably phospholipase A2, a T-REC or K-REC related protein, p-glucosidase, P-glucocerebrosidase. arylsulfatase A, Factor VIII, insulin-like growth factor 1 (IGF-1), surfactant protein A. surfactant protein B, aspartyl-P-glucosaminidase, acetyl-CoA a-glucosaminide, acetyl-CoA-arylamine N-acetyltransferase, N-acetylglucosamine-6-sulfatase, N-acetylglucosamine-1-Phosphotransferase, a-N-acetylglucosaminidase, acid ceramidase, aspartacylase, lysosomal acid lipase, acid sphingomyelinase, arylsulfatase B, ot-L-fucosidase, galactosylceramidase. galactocerebrosidase, P-galactosidase, protective protein/cathepsin A, a-glucoronidase, heparan N-sulfatase, a-hexosaminidase A, hyaluronidase-1, alpha-D-mannosidase, beta-mannosidase, alphaneuraminidase, beta-hexosaminidase A, beta-hexosaminidase B. palmitoyl-protein thioesterase, tripeptidyl peptidase I, Battenin, Ceroid-lipofuscinosis neuronal protein 5 (CLN5), Ceroid-lipofuscinosis neuronal protein 6 (CLN6), Ceroid-lipofuscinosis neuronal protein 7 (CLN7), Ceroid-lipofuscinosis neuronal protein 8 (CLN8), (Cathepsin D), cystinosin, cathepsin K, Sialin, Lysosome-associated membrane protein 2 (LAMP2), human growth hormone, follicle-stimulating hormone, erythropoietin, CD-I 9 or a granulocyte colony-stimulating factor (G-CSF) encoding gene.

15. (canceled)

16. (canceled)

17. (canceled)

18. (canceled)

19. A method for treating a disorder or disease, the method comprising delivering to a cell of a subject having the disorder or disease the composition of claim 12, wherein the disease or disorder is Pompe disease, Mucopolysaccharidosis Type I, Fabry′ disease, Mucopolysaccharidosis ty pe II, Mucopolysaccharidosis type IVA, Mucopolysaccharidosis type VI, Adrenoleukodystrophy, Severe combined immunodeficiency, Gaucher disease, metachromatic leukodystrophy (MLD), primary immune deficiency, hemophilia A, hemophilia B, IGF-1 deficiency, surfactant deficiency, Aspartylglycosaminuria, Sanfilipo syndrome, mucopolysaccharidosis type III, Sanfilippo Syndrome type IIId, I-cell disease, Schindler Disease. Farber disease (FD), Spinal muscular atrophy with progressive myoclonic epilepsy (SMA-PME), Canavan disease, Lysosomal acid lipase deficiency, Niemann-Pick disease, Mucopolysaccharidosis type 6, Fucosidosis, Krabbe disease. GM1 gangliosidosis, mucopolysaccharidosis type IVB (MPS IVB), or galactosialidosis. Sly disease, Mucopolysaccharidosis type III, Late-onset Tay Sachs, Hyaluronidase-1 deficiency, Alpha-mannosidosis, Beta-mannosidosis, Sialidosis, Stanhoff disease, Santavuori-Haltia disease, Jansky-Bielschowsky⁷ disease, Batten disease. Neuronal ceroid lipofuscinosis type 5, Neuronal ceroid lipofuscinosis type 6, Neuronal ceroid lipofuscinosis type 7. Neuronal ceroid lipofuscinosis type 8, Congenital cathepsin D deficiency, Cystinosis, Pycnodysostosis, Salla disease, Danon disease, and/or Alpha-1 antitrypsin deficiency.

20. (canceled)

21. (canceled)

22. Use of the composition of claim 12 for treating ameliorating or preventing a disorder or disease, comprising delivering to a cell of a subject having or at risk of having the disorder the composition of claim 12.

23. (canceled)

24. (canceled)

25. (canceled)

26. (canceled)

27. (canceled)

28. A method of treating a disease or disorder in a subject, wherein the method is an immunotherapy comprising delivering to the subject the modified cell of claim 9.

29. (canceled)

30. (canceled)

31. The method of claim 1, wherein the RNA molecule, or a DNA polynucleotide encoding the RNA molecule, comprising a guide sequence portion having at least 17 contiguous nucleotides of the nucleotide sequence set forth in any one of guides AAVS_s1 to AAVS_s39.

32. The RNA molecule, or a DNA polynucleotide encoding the RNA molecule, of claim 11, wherein the RNA molecule comprises a guide sequence portion comprising at least contiguous nucleotides of the nucleic acid sequence set forth in any one of guides AAVS_s1 to AAVS_s39.

33. A modified cell obtained by the method of claim 31.

34. A composition comprising the RNA molecule of claim 31, further comprising at least one CRISPR nuclease, or a DNA polynucleotide encoding the at least one CRISPR nuclease.

35. The composition of claim 34, wherein the at least one CRISPR nuclease comprises the nucleotide sequence of any one of the following OMNI nucleases: OMNI-50, OMNI-75, OMNI-79, OMNI-93, OMNI-103, OMNI-110, OMNI-127, OMNI-159, OMNI-175, OMNI-231, OMNI-269, OMNI-274, OMNI-281, OMNI-286, OMNI-291, OMNI-302, OMNI-308, and/or OMNI-366.
